(19) United States
(12) Patent Application Publication
(10) Pub. No.: US 2015/0223701 A1
(43) Pub. Date: Aug. 13, 2015

(54) BREATHING AND HEARTBEAT FEATURE EXTRACTION AND VICTIM DETECTION

(71) Applicant: California Institute of Technology, Pasadena, CA (US)

(72) Inventors: Hirad Ghaemi, Pasadena, CA (US); James P. Lux, Thousand Oaks, CA (US)

(73) Assignee: California Institute of Technology

(21) Appl. No.: 14/617,767

(22) Filed: Feb. 9, 2015

Related U.S. Application Data
(60) Provisional application No. 61/938,064, filed on Feb. 10, 2014.

(51) Int. Cl. A61B 5/0205; A61B 5/00

(52) U.S. Cl. CPC A61B 5/0205; A61B 5/725; A61B 5/7278; A61B 5/7246; A61B 5/7203; A61B 5/7275; A61B 5/7282; A61B 5/0507

(57) ABSTRACT

Systems and methods for detecting biometrics using a life detecting radar are disclosed. Life detecting radars can include transmit antennas configured to transmit continuous microwave (“CW”) radio signals that reflect back upon making contact with various objects. The life detecting radars can identify victims by analyzing data with respect to a victim's breathing and heartbeat patterns. In some embodiments, to identify victims, the return signal may be split into a heartbeat band and a breathing band using bandpass filtering. The life detecting radar system may perform parameter estimation for the breathing band and/or the heartbeat band using a non-least squares process (NLS). The life detecting radar system may analyze breathing and heartbeat results based on the heartbeat FM frequency and the breathing center frequency to identify victims.

### CROSS-REFERENCE TO RELATED APPLICATIONS

The current application claims priority to U.S. Provisional Patent Application No. 61/938,064 filed Feb. 10, 2014, the disclosure of which is incorporated herein by reference.

### FEDERAL FUNDING SUPPORT

The invention described herein was made in the performance of work under a NASA contract, and is subject to the provisions of Public Law 96-517 (35 USC 202) in which the Contractor has elected to retain title.

### FIELD OF THE INVENTION

The present invention generally relates to radars and more specifically to systems and methods for detecting biometrics using radars.

### BACKGROUND

Biometrics refer to the quantifiable data (or metrics) related to human characteristics and traits. The quantifiable metrics can be gathered using various sensors and the collected data processed to identify individual persons. Typically, biometric identifiers can be categorized as physiological and/or behavioral characteristics. Generally, physiological characteristics are related to the shape of the body and can include (but not limited to) fingerprint, palm print, DNA, and scent. In contrast, behavioral characteristics relate to a pattern of behavior and include (but not limited to) gait, voice, and typing rhythm. Biometric identifiers can also include characteristics that are more subtle such as breathing patterns and heart rates.

### SUMMARY OF THE INVENTION

Systems and methods in accordance with embodiments of the invention use radar to detect the location of living people. One embodiment includes an integrated microwave sensor module that includes a transmitter unit with a variable frequency microwave source connected to at least one transmitter unit amplifier. The variable frequency microwave source is configured to generate at least one continuous wave (“CW”) transmit signal based upon at least one frequency control signal received from a microcontroller unit. The at least one transmitter unit amplifier is configured to receive and amplify the at least one CW transit signal. The integrated microwave sensor module also includes a receiver unit configured to receive at least one return signal and utilize a cancellation path to cancel contributions to the return signal that are not the result of reflections from a target. The microcontroller unit is configured to communicate with the transmitter and receiver units. The microcontroller unit includes a processor, a memory containing a microcontroller application. The microcontroller application configures the processor to split the return signal into a heartbeat band and a breathing band using bandpass filtering, perform parameter estimation for the breathing band using a non-least squares process (NLS), perform parameter estimation for the heartbeat band using an NLS process, analyze breathing and heartbeat results based on the heartbeat FM frequency and the breathing center frequency, and output detected targets based on analysis.

In a further embodiment, a NLS process fits a complex input to a frequency modulated (FM) model.

In another embodiment, the microcontroller application further configures the processor to low pass filter and decimate the return signal.

In a still further embodiment, the microcontroller application further configures the processor to remove a linear trend from the return signal using a linear least square fitting in the data.

In a yet further embodiment, the microcontroller application further configures the processor to remove signals that are out of band.

In another embodiment again, the microcontroller application further configures the processor to identify and remove 2^{nd} and 3^{rd} harmonics from a list of detected frequencies of the breathing band.

In another embodiment, the microcontroller application further configures the processor to remove harmonics of breathing signals that appear in the heart band.

In yet another embodiment, the microcontroller application further configures the processor to remove out of band heartbeat signals from the heart band by removing targets whose center frequency is out of an assigned bandwidth.

In another embodiment, the microcontroller application further configures the processor to remove targets whose FM frequency is out of a particular FM frequency range.

In a still yet further embodiment, the microcontroller application further configures the processor to remove targets whose relative amplitude with respect to a maximum in the heartbeat band is below a certain threshold based on a dynamic range for detected targets.

In still another embodiment again, the microcontroller application further configures the processor to remove targets whose relative amplitude with respect to a maximum in the breathing band is below a certain threshold based on a desired dynamic range for detected targets.

In yet another embodiment, the microcontroller application further configures the processor to remove heart signals whose relative amplitude with respect to breathing is large within a certain threshold.

In another further embodiment, the microcontroller application further configures the processor to match breathing results with heartbeat results based on the heartbeat FM frequency and breathing center frequency.

In a still further embodiment, the microcontroller application further configures the processor to calculate reliability factors for the heartbeat band and the breathing band by using corresponding signal to noise ratio “SNR” values.

In another embodiment, the microcontroller application further configures the processor to compare a plurality of signals received from a plurality of receivers to identify false targets.

In yet another embodiment, the microcontroller application further configures the processor to compare results from each of a plurality of received signals to identify false targets.

An embodiment of the method of the invention includes: propagating at least one beam using a continuous wave transmit signal set at a plurality of frequencies, where the at least one beam illuminates at least one sensing area using at least one transmit unit, receiving a return signal associate with reflections of the at least one transmit signal from objects within the at least one sensing area using at least one receive antenna, receiving the return signal from the at least one receive antenna using a life detecting radar system, splitting the return signal into a heartbeat band and a breathing band using bandpass filtering, performing parameter estimation for the breathing band using a non-least squares process (NLS), performing parameter estimation for the heartbeat band using an NLS process, analyzing breathing and heartbeat results based on the heartbeat FM frequency and the breathing center frequency, and outputting detected targets based on analysis.

In a further embodiment, a NLS process fits a complex input to a frequency modulated (FM) model.

In still another embodiment, the method applies a low pass filter to the return signal.

In yet another embodiment, the method removes a linear trend from the return signal using a linear least square fitting in the data.

### BRIEF DESCRIPTION OF THE DRAWINGS

FIG. 1 is a system diagram of a life detecting radar (“FINDER”) in accordance with an embodiment of the invention.

FIG. 2 illustrates an antenna unit in accordance with an embodiment of the invention.

FIG. 3A illustrates a FINDER utilizing a single beam for detection in accordance with an embodiment of the invention.

FIG. 3B illustrates a FINDER utilizing multiple beams for detection in accordance with an embodiment of the invention.

FIG. 4A and 4B illustrate FINDER units utilizing multiple frequencies in accordance with an embodiment of the invention.

FIG. 5A illustrates a process for signal processing used in target detection and parameter estimation for a FINDER system in accordance with an embodiment of the invention.

FIG. 5B illustrates an example of the high level data flow of a FINDER system in accordance with embodiments of the invention.

FIG. 5C illustrates an example of a detailed process for identifying victims in accordance with an embodiment of the invention.

FIG. 6 illustrates an example of typical values stored for various variables in accordance with an embodiment of the invention.

FIG. 7A illustrates an example of a table with a set of values for an FM signal in accordance with an embodiment of the invention.

FIG. 7B illustrates a graph of an example of a sample spectrum in accordance with an embodiment of the invention.

FIG. 7C illustrates a graph of an example heart rate in accordance with an embodiment of the invention.

FIG. 8 illustrates an example of a sample RDF file with explanations for the various parameters in accordance with an embodiment of the invention.

FIG. 9 illustrates an overall block diagram of the initial multi-stage decimation low pass filter in accordance with an embodiment of the invention.

FIG. 10 illustrates a process for processing a channel signal in accordance with an embodiment of the invention.

FIG. 11 illustrates a table that lists a process of some embodiments for processing a signal.

FIG. 12 illustrate a table that lists s process of some embodiments for processing a signal.

FIGS. 13A-D illustrate an example of stages of the overall filter response used by a FINDER system in accordance with an embodiment of the invention.

FIGS. 14A-B illustrate an example of stages of the overall filter response used by a FINDER system in accordance with an embodiment of the invention.

FIG. 15 illustrates an example image in accordance with embodiments of the invention.

FIG. 16 illustrates an example of the data flow of the decimation to a lower sample rate and trimming of the ends of the time series by a FINDER system in accordance with embodiments of the invention.

FIG. 17 shows an example of a sample of the bandpass characteristics for the two bands.

FIG. 18 illustrates a table that shows sample data after the initial relaxation process performed on the breathing band data

FIG. 19 illustrates an example of a harmonic rejection process in accordance with an embodiment of the invention.

FIG. 20 illustrates a table with an example of sample data after removal of the harmonics in accordance with an embodiment of the invention.

FIG. 21 illustrates a table with an example of a set of initial estimated values in accordance with an embodiment of the invention.

FIG. 22 illustrates an example of the initial estimated values for both breathing and heartbeat bands.

FIG. 23 illustrates an example of the corresponding list of values after an elimination process in accordance with an embodiment of the invention.

FIG. 24 illustrates an example of results after a selection process in accordance with an embodiment of the invention.

FIG. 25A illustrates an example of results for breathing model parameters after a selection process in accordance with an embodiment of the invention.

FIG. 25B illustrates an example of results for heartbeat band parameters after a selection process in accordance with an embodiment of the invention.

FIG. 26 illustrates an example of remaining heartbeat values after a selection process in accordance with an embodiment of the invention.

FIG. 27A illustrates an example of parameters of the FM model for the breathing band in accordance with an embodiment of the invention.

FIG. 27B illustrates an example of parameters of the FM model for the heartbeat band in accordance with an embodiment of the invention.

FIG. 28A illustrates a table with an example of the corresponding results for breathing model parameters after an elimination process in accordance with an embodiment of the invention.

FIG. 28B illustrates a table with an example of the corresponding results for heartbeat model parameters after an elimination process in accordance with an embodiment of the invention.

FIG. 29 illustrates an example of variables that may be used to output information in accordance with embodiments of the invention.

FIGS. 30A-E illustrate an example of the effective bystander rejection azimuth range or beam width for various comparison thresholds from −6 dB to +6 dB win accordance with embodiments of the invention.

FIG. 31 illustrates an example of the approximate “beam width” as a function of an RDF parameter in accordance with embodiments of the invention.

FIGS. 32A-F illustrate an example that shows the detected targets at each beam prior to multi-beam processing.

FIG. 33 illustrates an example of sample detected targets and output from multi-beam processing in accordance with an embodiment of the invention.

FIG. 34 illustrates an example of a true victim list after multi-beam processing in accordance with embodiments of the invention.

FIG. 35 illustrates an example of a structure of software modules and their corresponding subroutines as well as functionality used for signal processing in accordance with embodiments of the invention.

### DETAILED DESCRIPTION OF THE DRAWINGS

Turning now to the drawings, systems and methods for detecting biometrics using a life detecting radar in accordance with embodiments of the invention are disclosed. In many embodiments, life detecting radars include one or more transmit antennas configured to transmit continuous microwave (“CW”) radio signals that reflect back upon making contact with various objects. In many embodiments, the signal is systematically varied in frequency to provide a signal that is essentially continuous with short gaps between transmissions at different frequencies. In several embodiments, the reflected return signals are received by one or more receive antennas and processed to detect one or more targets. In various embodiments, the received signal can include a static (i.e. constant phase) signal corresponding to reflections from objects that do not move. The received signal can also include a phase varying signal that corresponds to reflections from a living target having measurable biometrics including (but not limited to) breathing patterns and heartbeats. In various embodiments, clutter (i.e. portions of the signal not corresponding to target reflections) is removed and the remaining portions of the received signal are analyzed for target detection. In a variety of embodiments, multiple antennas and multiple frequencies are utilized to create so-called sensing areas.

In one application, a life detecting radar (“FINDER”) system can be utilized to locate victims buried within disaster rubble. In many embodiments, a CW radar is utilized to detect physical changes in a target such as (but not limited to) motion due to heartbeats and/or breathing. In many embodiments, targets can be detected by taking the raw radar data and performing range processing where stepped frequency data is taken and an inverse Fast Fourier Transform (FFT) applied to turn the frequency domain data into an equivalent time domain profile. In several embodiments, target Identification can be attempted to find unique targets in one or more beam and range bins by splitting the signal into a heart rate band and a breathing band and analyzing the relationship between these bands based on the typical Respiratory Sinus Arrhythmia (RSA) relationship. This analysis relies on the fact that while a given target's heart rate and breathing rate may vary, the general shape of their microwave cardiogram (“MCG”) waveform does not (it merely stretches and shrinks). In some embodiments, features in the form of respiration and heartbeats are extracted from the signal data and victims are identified from the feature list. In many embodiments, there is one set of data for each potential victim and data that identifies which beams/ranges that signal appears in. The set of data concerning each victim or target can also include data about the variability of that target.

In some embodiments, the FINDER system compares the detected targets in each of the received channels, and if the same target (e.g., heart rate, respiration) is detected in more than one channel, and is stronger in the back or side beams, then it may be assumed that the target is a bystander and is thus removed from the victim list.

Although FINDER is described in detail below as applied to detecting victims buried in rubble, it can have various other applications including (but not limited to) detecting prisoners barricaded in a prison, suspects hiding in farm fields or houses, as well as being used as a form of diagnostic or biometric measurement instrument. Finder systems for detecting biometrics of and/or identifying a target in accordance with embodiments of the invention are further discussed below.

### Life Detecting Radar (“FINDER”) Systems

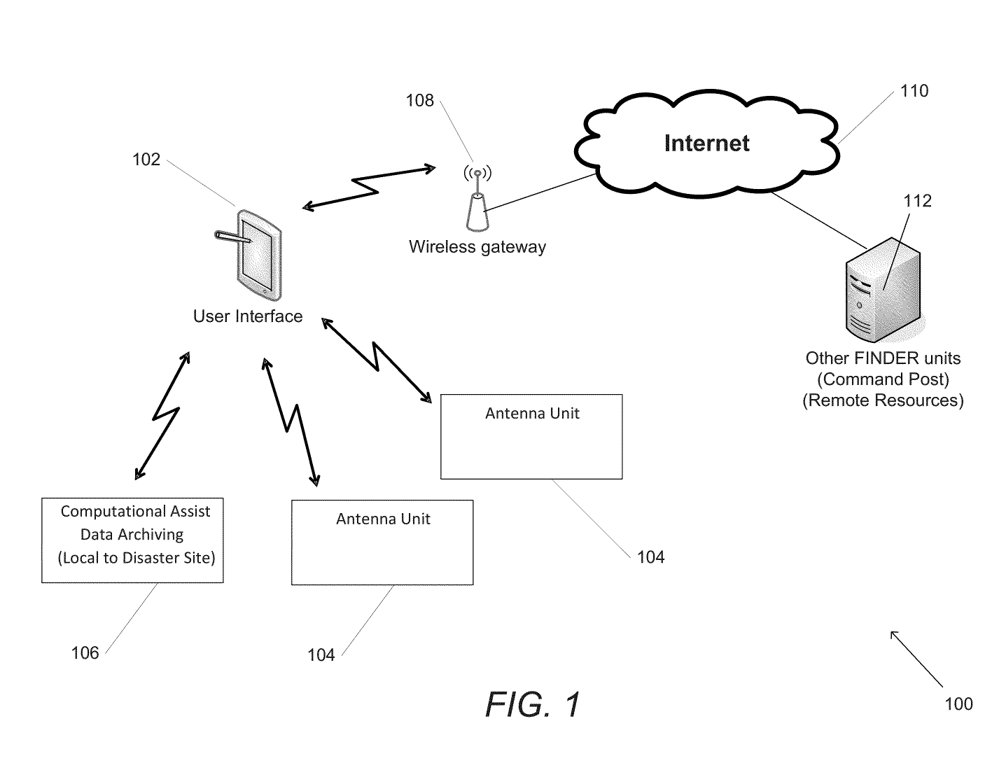

FINDER (acronym for Finding Individuals for Disaster and Emergency Response) systems can be utilized to detect biometrics (i.e. physiological characteristics) of various targets. A FINDER system in accordance with an embodiment of the invention is illustrated in FIG. 1. The system 100 includes a user interface 102 configured to wirelessly connect and control at least one antenna unit 104, where the antenna unit transmits and receives radio signals as further described below. In several embodiments, the user interface 102 can also wirelessly connect to various other units including (but not limited to) computational assist units and data archiving units 106. In many embodiments, the user interface 102 can communicate wirelessly with a cellular data network 108 (i.e. wireless gateway) to connect to the Internet 110. Utilizing the Internet 110, the user interface 102 can access additional units including (but not limited to) a command post and other remote resources 112. Although described as separate units, in a variety of embodiments, the user interface 102 and the various units 104, 106 can be one physical unit communicating with each other via a direct network link or other means of data communication. FINDER systems that can be used to detect biometrics are described in U.S. Patent Publication No. 2014/0316261A1, entitled “Life Detecting Radars”, filed Apr. 18, 2014 and published on Oct. 10, 2014, the disclosure of which is hereby incorporated by reference in its entirety

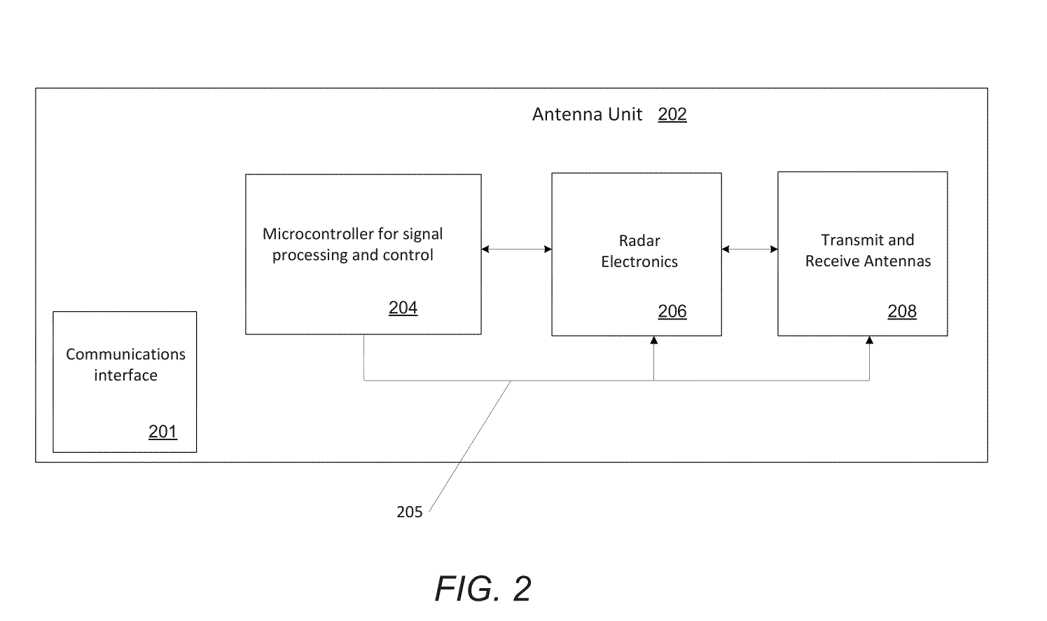

As described above, a FINDER system can include one or more antenna units configured to transmit radio signals including (but not limited to) continuous wave signals and to receive reflected return signals. An antenna unit in accordance with an embodiment of the invention is illustrated in FIG. 2. The antenna unit 202 includes a microcontroller (and/or an embedded PC) 204 that can send control signals 205 to radar electronics 206 and antennas 208 in connection with the microcontroller 204. In various embodiments, the radar electronics themselves can be microcontrollers. In additional embodiments, radar electronics 206 can be incorporated with the transmit antenna 208 (i.e. transmit module). Likewise, radar electronics 206 can be incorporated with the receive antenna 208 (i.e. receive module). In several embodiments, a communications interface 201 can be used to send and receive information or communicate with other antenna units. Communications interface 201 may be wired or wireless. In many embodiments, the antennas 208 include transmit antennas for transmitting radio signals as further discussed below. The antennas 208 can also include receive antennas for receiving return signals that include reflections from various physical objects in the search area as further discussed below. In various embodiments, the received signal is stored as digital radar data and transmitted to the microcontroller (and/or an embedded PC) 204 for signal processing as further discussed below.

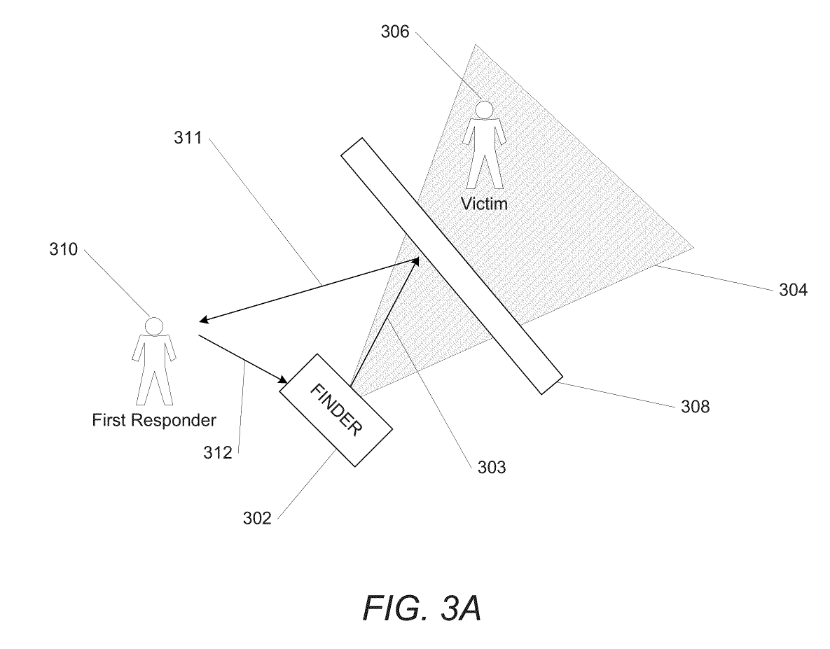

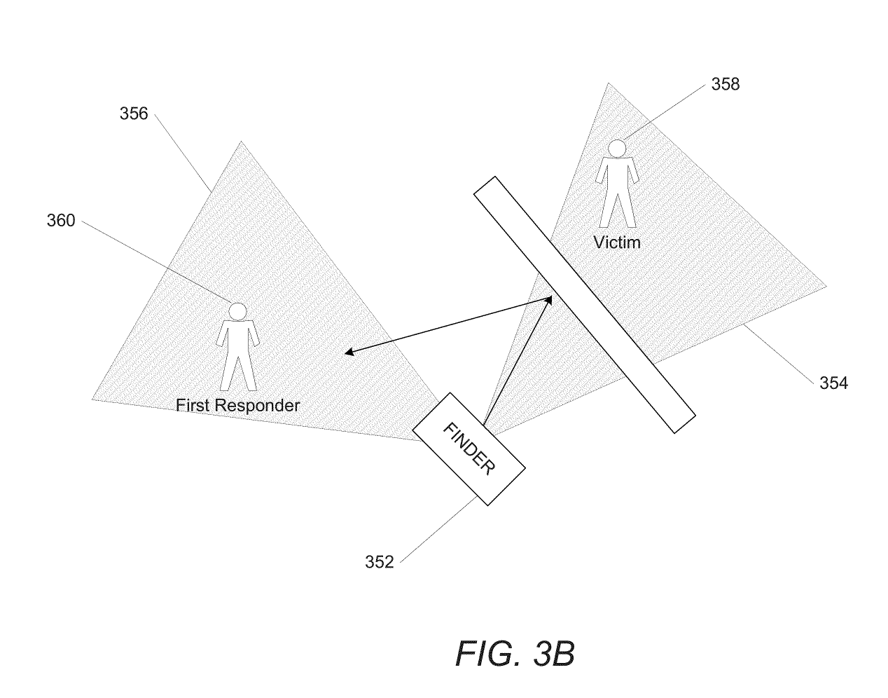

The ability for a FINDER system to form multiple beams can improve target identification and separation. A FINDER system utilizing a single beam for detection in accordance with an embodiment of the invention is illustrated in FIG. 3A. The FINDER system 302 transmits signals to illuminate a single beam 304 to detect a victim 306 who is surrounded by rubble. Often in real life search scenarios, various objects 308 reflect the transmit signal 303 in undesired directions resulting in unwanted return signals. Further, search personnel (“first responders”) 310 can also cause return signals 312 and be misidentified as victims. As illustrated, the transmitted signal 303 is reflecting off various objects 308, and then that reflection 311 is reflecting off the bystander 310, eventually ending up at the FINDER 302. In many embodiments, the beam is not ideal with sharp edges meaning even though the beam 304 is generally directed in a particular direction, signals will be transmitted and received in all directions, at reduced amplitudes.

The use of multiple beams can increase detection accuracy and sensitivity. A FINDER system utilizing multiple beams for detection in accordance with an embodiment of the invention is illustrated in FIG. 3B. The FINDER 352 can form multiple beams 354 and 356 as further discussed below. The first beam 354 can detect the victim 358 while the second beam 356 can eliminate the first responder 360 as a possible victim as further discussed below. In addition, the ability to simultaneously “view” the search area in multiple directions can be useful. For example, being able to look in multiple directions at the same time allows rejection of phantom victims in the search area that are really just reflections from someone standing behind the FINDER antenna unit or next to the search area. In many embodiments, FINDER systems can be designed such that the basic radio frequency (“RF”) signal chain is readily scalable to multiple beams and locations.

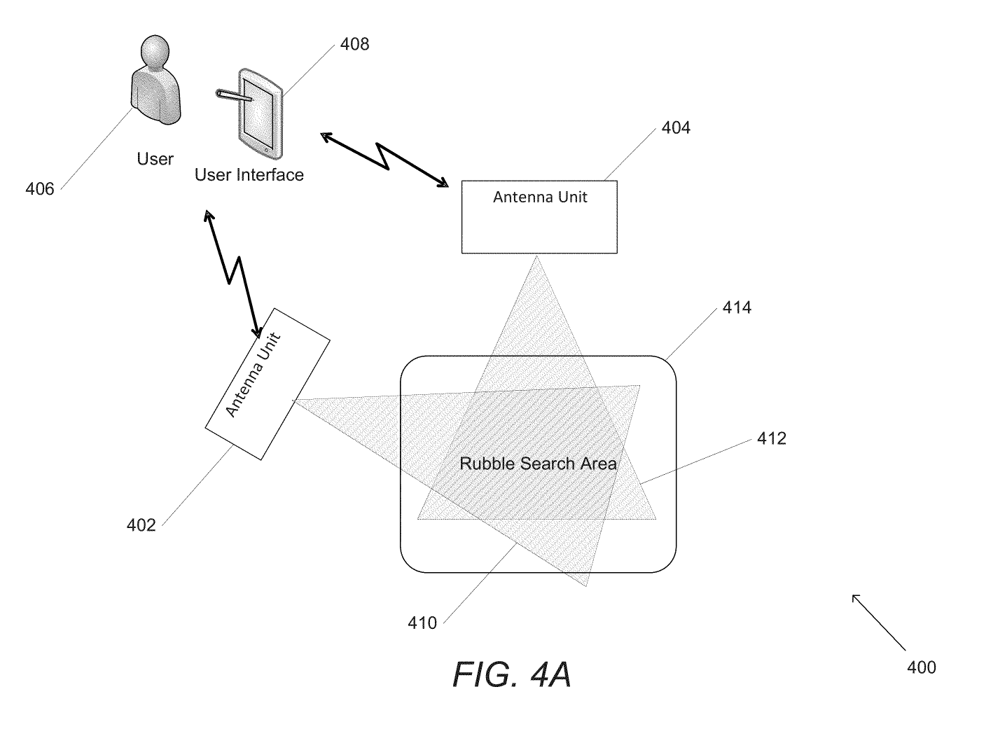

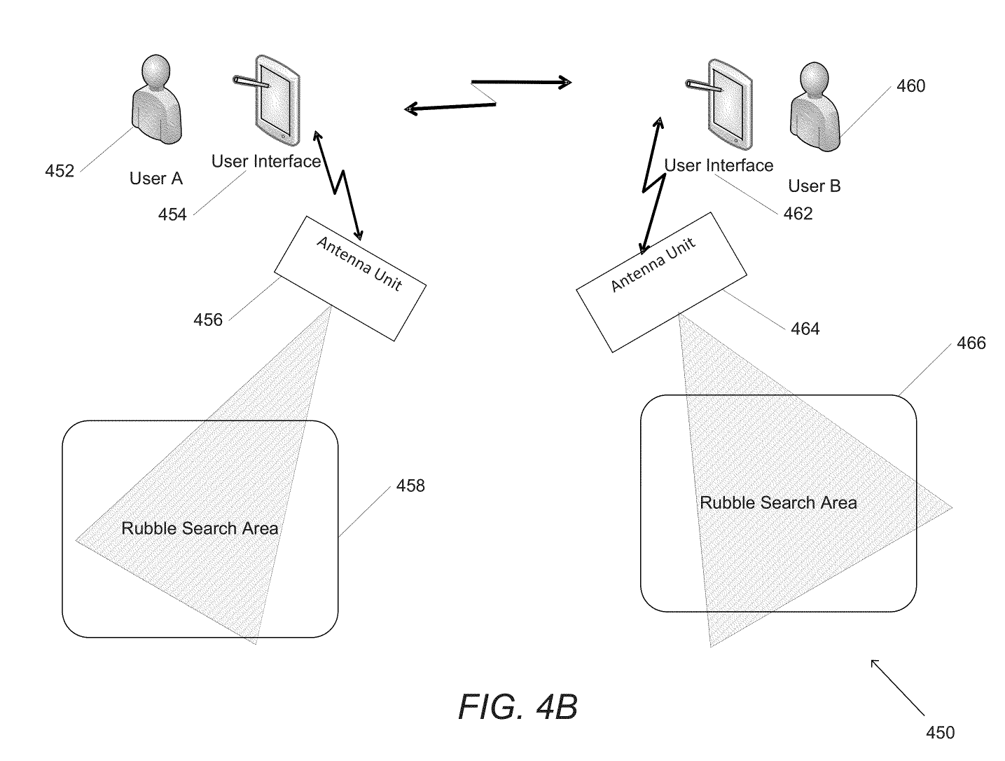

In addition to multiple beams, FINDER systems can utilize multiple frequencies in an allocated bandwidth. A FINDER system employing multiple frequencies can avoid interference by signals from other sources and/or not interfere with other systems by using a different frequency from such other systems. The use of multiple frequencies in accordance with an embodiment of the invention is illustrated in FIGS. 4A-B. The search scenario 400 illustrates two antenna units 402 and 404 being controlled by a single user 406 via a single user interface 408. The antenna unit 402 transmits a transmit signal to illuminate a beam 410 at a first frequency while antenna unit 404 transmits a separate transmit signal to illuminate a second beam 412 at a second frequency. Both beams 410 and 412 are transmitted to the same rubble search area 414 without interfering with each other because the two transmit signals operate at different frequencies. FINDER systems utilizing multiple frequencies to illuminate two separate rubble search areas at the same location in accordance with an embodiment of the invention is illustrated in FIG. 4B. The search scenario 450 illustrates User A 452 utilizing a user interface 454 that communicates with an antenna unit 456 to illuminate a rubble search area 458 utilizing a first frequency. At the same location, User B 460 can utilize a user interface 462 to communicate with an antenna unit 464 to illuminate a rubble search area 466 using a second frequency. Again, the use of multiple frequencies allow for the FINDER systems to avoid interfering with each other while operating in the same location. Furthermore, the detection of victims or targets can be enhanced by combining the outputs of multiple FINDER systems to collect data concerning a target from multiple directions. In several embodiments, synchronized data recording can be utilized to enable the detection of matching time varying signals such as (but not limited to) respirations and heart beats in signals received by different antennas and/or FINDER systems.

Although specific FINDER systems for detecting victims are discussed above with respect to FIGS. 1-4B, any of a variety of FINDER systems for detecting victims as appropriate to the requirements of a specific application can be utilized in accordance with embodiments of the invention. Signal processing for victim detection in accordance with embodiments of the invention are discussed further below.

### Signal Characteristics and Signal Processing

FINDER systems utilize the principle of looking for small phase changes in a CW signal reflected from a victim. As victims breath, their bodies move slightly (in particular, their chest walls on the order of 1 cm), and similarly, their heartbeats cause the abdominal surface and many other portions of the human body to move (on the order of 1 mm). The moving body causes reflections of transmit signals with varying phases (i.e. phase change). The detected phase change by receive antennas can form the basis of the so-called microwave cardiogram (“MCG”). Typically, each person has a unique MCG which varies depending on his orientation relative to the sensor, and, their physiological state. The uniqueness of a MCG allows for the separation of combined MCGs from multiple targets (statistical analysis shows that it is unlikely that two people would have exactly the same heart rate, and even if the average rate were the same, the beat to beat variability is a random process, causing the two sequences to be uncorrelated). However, in real search scenario, there may be a multitude of other objects besides the victim reflecting a microwave signal back to the receiver, including (but not limited to) the rubble surrounding the victim, and objects near the radar. Typically, such signals are reflected from objects that are not moving and thus the phase stays relatively constant/static. The return signal that a radar receiver detects is typically a combination of a strong static signal component (corresponding to reflections from non-moving objects) that is unchanging with a weaker time varying signal component (corresponding to a victim). In terms of level, the static signal component that is received by the radar is typically on the order of 20 dB weaker than the transmitted signal, while the time varying return signal reflected off a victim is typically 60-100 dB (or more) weaker. The dominant reason for the weaker signal from the victim is the scattering of the signal in the rubble, more than the bulk attenuation in the rubble material.

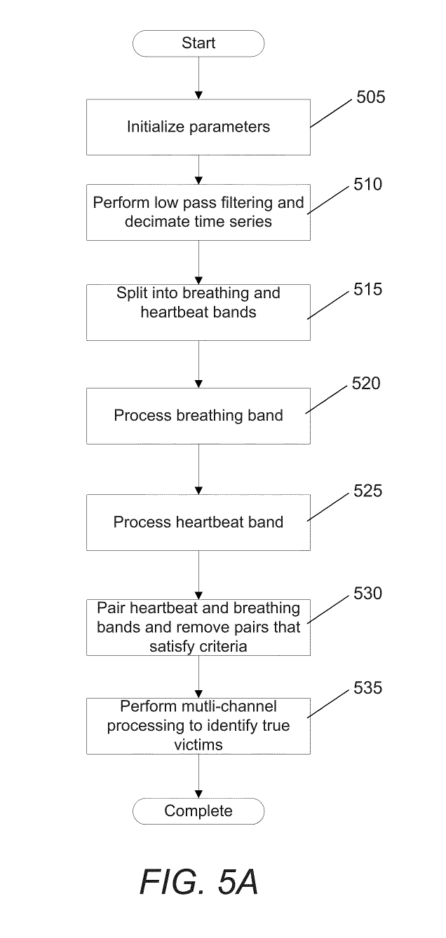

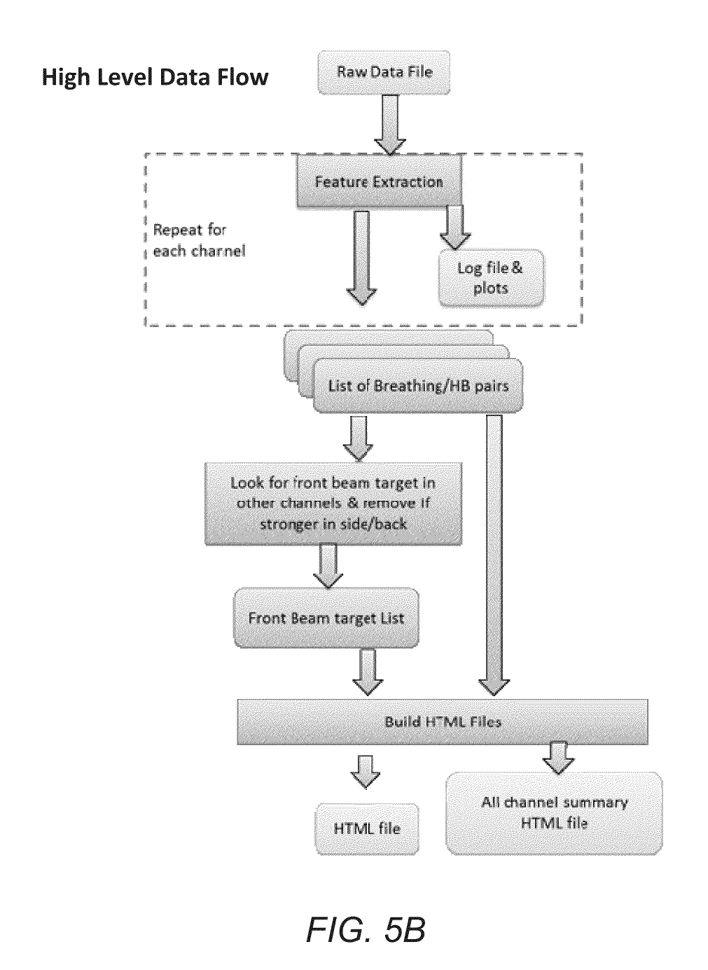

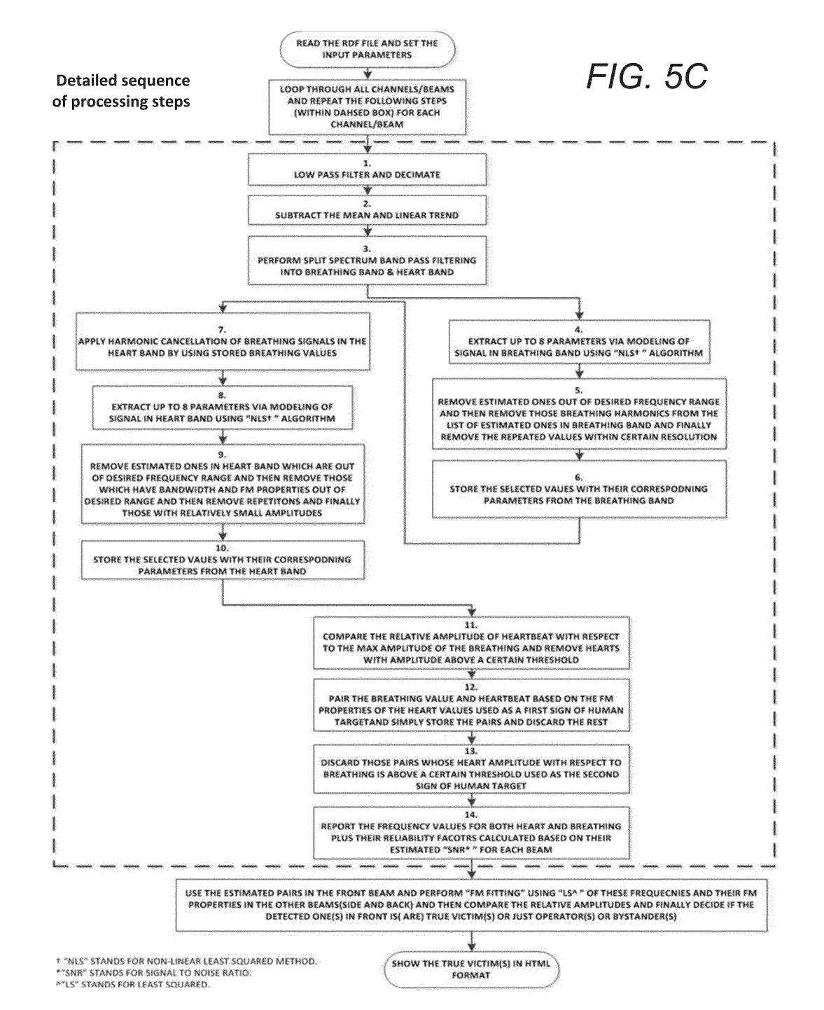

A process for signal processing used in target detection and parameter estimation for a FINDER system in accordance with an embodiment of the invention is illustrated in FIG. 5A. The process initializes (at 505) one or more parameters. As described in detail in section “Preprocessing Setup and Parameter Initialization”, the parameters may be initialized using an input file, such as a Radar Definition File “RDF” file, that specifies a set of parameter values. In other embodiments, the parameters may be specified using a variety of different mechanisms, including passing them via command line parameters, or through an operating system mechanism such as, for example, a registry for the Windows™ operating system. Although the description below describes using an RDF file to set the parameters, any of a number of different mechanisms may be used as appropriate to the requirements of specific applications to set parameters in accordance with embodiments of the invention.

The parameter set may include parameters related to the Radar data and its configuration, parameters needed for configuring the multi-stage decimated low-pass filter, parameters for both band pass filters (split spectrum filters) used for separating out bands related to breathing and heartbeat, parameters used by a non-linear least squared (“NLS”) process to extract one or more FM modeling parameters in both the breathing and heartbeat band, and timing and decision making parameters used in target identification, removal of undesired values and separating true victim(s) from bystanders and/or operators.

The input file may also specify a set of flags that may be used to determine whether or not to execute certain signal processing computations.

The process performs (at 510) low pass filtering and decimates the original input time series. As described in detail in section “1. Low Pass Filter and Decimate” below, the original input time series in some embodiments may be filtered and decimated to a certain sampling rate. In some embodiments, the sampling rate is 20 Hz. Although specific sampling rates and decimation techniques have been disclosed, FINDER systems for detecting victims may use different sampling rates and/or decimation techniques as appropriate to the requirements of specific applications in accordance with embodiments of the invention.

In some embodiments, either a Finite Impulse Response (“FIR”) or an Infinite Impulse Response (IIR) filter and corresponding decimators may be used. In some embodiments, a series of Chebyshev Finite Impulse Response filters and corresponding decimators may be used. The corresponding parameters to configure the filter may be specified in the input RDF file.

The process splits (at 515) out breathing and heartbeat bands. As described in detail in section “3. Split into Breathing and Heartbeat Bands”, the single time series may be split into two time series/signals, a breathing signal and a heartbeat signal. In some embodiments, the single signal is split using two BPF filters. Although a specific signal splitting technique has been disclosed, any of a variety of signal splitting techniques and/or filters may be used as appropriate to the requirements of specific applications to obtain the different processing bands in accordance with embodiments of the invention.

The process processes (at 520) the breathing band. As described in detail in sections “5. Parameter Estimation for Breathing Band” and “6. Store Breathing Values”, the processing of the breathing band may include parameter estimation of the breathing band using a nonlinear least squares process (“NLS”). The NLS process may fit the complex input to a FM model and the output may include a number of detected targets that fit the model, among various other information. In some embodiments, a relation process similar to the process described by Hirad Ghaemi is used to solve the NLS problem for estimating the number of targets and their corresponding features. See Hirad Ghaemi, “Synthetic Aperture Weather Radar,” Master Thesis, Chalmers University of Technology, Goteborg, Sweden, 2008 available at http://elib.dlr.de/54507/1/MScThesis_H.Ghaemi.pdf.

The process processes (at 525) the heartbeat band. Section “7. Remove Breathing Harmonics in Heartbeat Band” through Section “10. Store Heartbeat Values” described below provide more details with respect to the processing of the heartbeat band in accordance with many embodiments of the invention.

The process pairs (at 530) the heartbeat and breathing bands and removes pairs that satisfy a criteria. Sections “11. Remove heartbeat signals with relatively large amplitude” through Section “14. Calculate Reliability Scores” described below provide more details with respect to the pairing of the heartbeat and breathing bands in accordance with many embodiments of the invention.

The process performs (at 535) multi-channel processing to identify victims. Section “Multichannel processing” described below provides more details with respect to the multi-channel processing.

An example of the high level data flow of a FINDER system in accordance with embodiments of the invention is illustrated in FIG. 5B. Furthermore, an example of a detailed process for identifying victims in accordance with an embodiment of the invention is illustrated in FIG. 5C.

As illustrated in FIG. 5B, the FINDER system may receive a raw data file that may include signal data and repeats, for each channel, feature extraction and/or log file & plots. Based on the feature extraction, the FINDER system may generate a list of breathing band and heartbeat band pairs and may use the pairs to generate a file of the list of detected targets. The FINDER system may also use the list of breathing and heartbeat band pairs to look for a front beam target in other channels and the FINDER system may remove the target if the signal data is stronger in the side and/or back channels. The FINDER system may generate a front beam target list and provide this information for further processing. Although FIG. 5B illustrates an example of a high level data flow of a FINDER system, any of a variety of data flow architectures may be utilized as appropriate to the requirements of specific applications in accordance with embodiments of the invention.

Signal propagation characteristics in accordance with an embodiment of the invention are described below. Although specific processes for signal processing used in target detection and parameter estimation for a FINDER system in accordance with an embodiment of the invention are described above, any of a variety of processes may be utilized for signal processing used in target detection and parameter estimation as appropriate to the requirements of specific applications in accordance with embodiments of the invention.

### Model Description

As described above, in many embodiments, a key part of the target identification is the fitting of a generalized model of the reflected signal. The model may be previously developed using empirical test data obtained from a laboratory and/or field test sites, and that closely matches the expected form from the underlying physiological phenomenon.

In some embodiments, a based form of the model is given as equation (1A), below:

D sinc(B(t−T))e^{i2πf}^{_{c}}^{t}e^{ih cos(2πf}^{ₘ}^{t+φ)} (1A)

The significance of the various components is as follows.

D is a complex value, and is the overall amplitude and phase of the target and is primarily affected by the distances to the target and the intervening material. Since the target is not moving, this term is essentially constant.

The B & T parameters provide for slow changes over the typical 30 second data epoch are accounted for in the sinc(B(t-T)) term, which provides for a general rise and fall, at least for the data that has been collected to date. This amplitude modulation also results in slight broadening of the spectral line (sinc( )is the amplitude spectrum of a rectangular pulse), but given typical B parameters of 0.001 to 0.004 for breathing and 0.02 for heart rate, this effect is not great. Certain embodiments may model the random 1/f characteristic of both the respiration and heart rate rhythms.

In many embodiments, the base heart rate during the epoch is represented by the exp(j2πf_{c}^{t}) term, and in humans, fc ranges from around 40-110 beats/min or 0.66-1.8 Hz. All mammals typically exhibit a modulation of the heart rate linked to respiration known as respiratory sinus arrhythmia (RSA), and this is modeled by the exp(jhcos(2πfₘt+φ)) term. More discussion of RSA can be found in the literature, notably “Respiratory sinus arrhythmia in humans: how breathing pattern modulates heart rate” by Hirsch and Bishop, Am J Physiol. 1981 October: 241(4): H620-9.

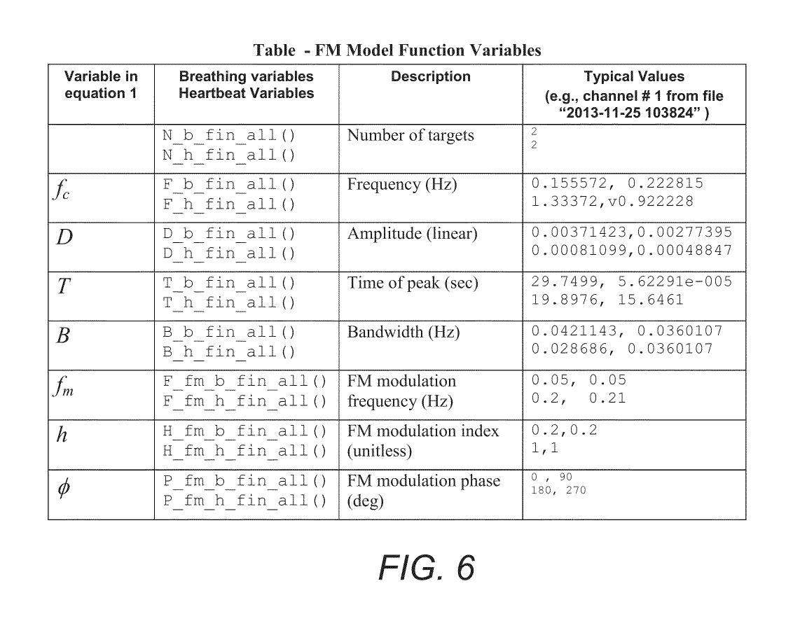

An example of typical values stored for various variables in accordance with an embodiment of the invention is illustrated in FIG. 6. Although FIG. 6 illustrates an example of an input file for configuring a FINDER system, any of a variety of input files with different parameter values as appropriate to the requirements of specific applications may be used to configure FINDER systems in accordance with embodiments of the invention.

### Expected Detected Signals

The classic frequency modulated (FM) signal is represented by equation (2A), below:

$$y ( t ) = {A}_{c} cos ( 2 π {f}_{c} t + \frac{{f}_{Δ}}{{f}_{m}} cos ( 2 π {f}_{m} t ) ) \qquad ( 2 A )$$

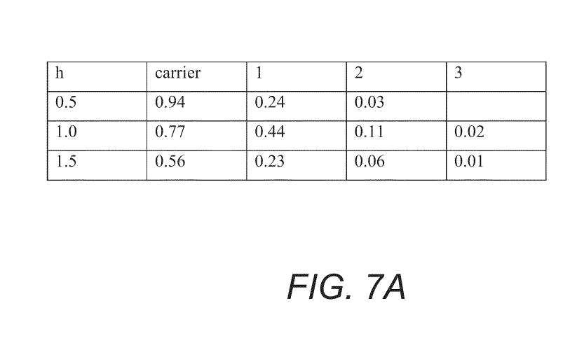

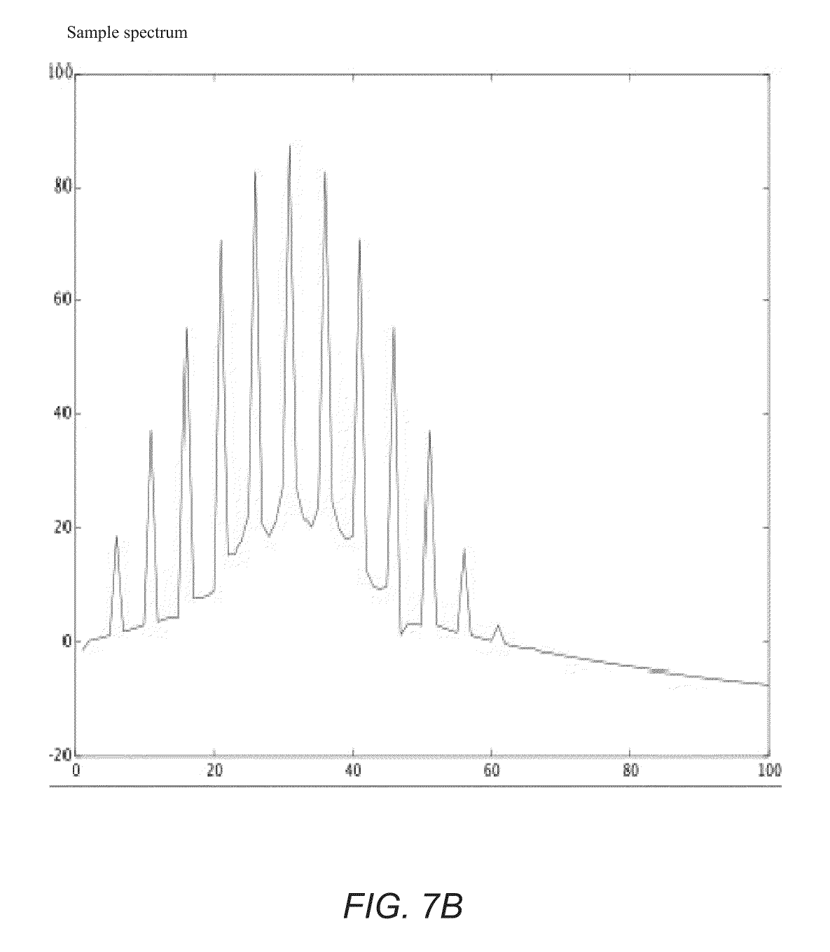

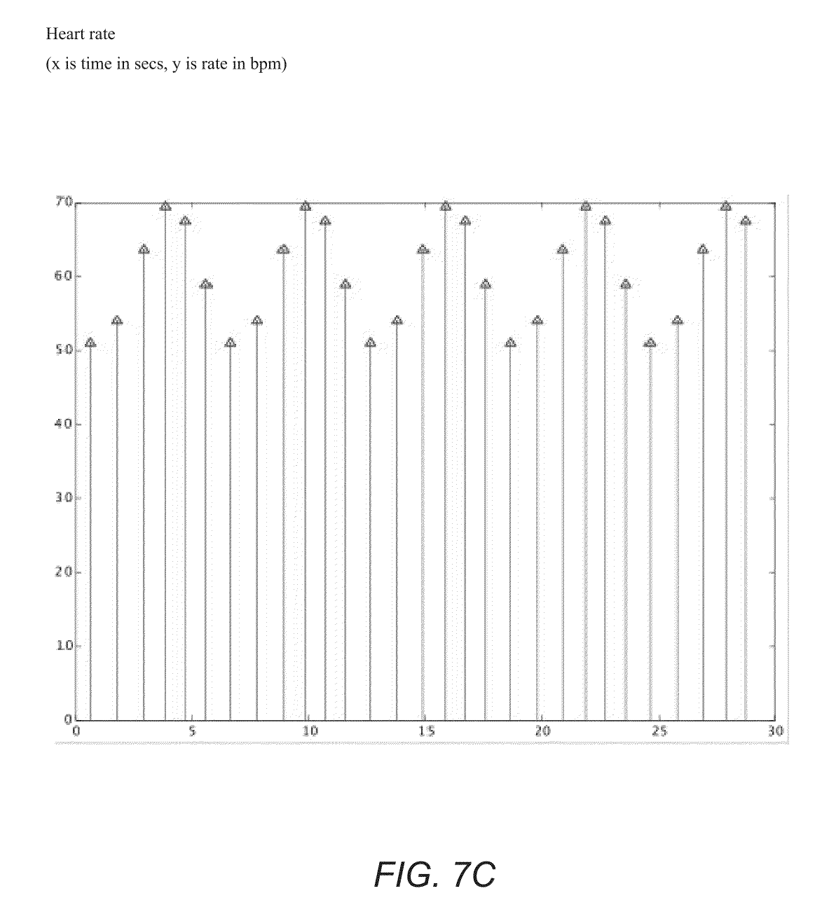

The fΔ/fₘ term is usually referred to as h, the modulation index, and in the typical human case, fΔ is on the order of 20 beats/minute or 0.32 Hz. This is quite close to double the respiration rate (fₘ), so h is close to unity (the variation is above and below the center frequency, so the RSA is twice fΔ). For h of 0.5 to 1.5, expect from 2 to 4 sideband tones according to Carson's rule, although the last few are typically quite low level as illustrated in the table illustrated in FIG. 7A. A graph of the sample spectrum is illustrated in FIG. 7B and the heart rate is illustrated in FIG. 7C.

### Preprocessing Setup and Parameter Initialization

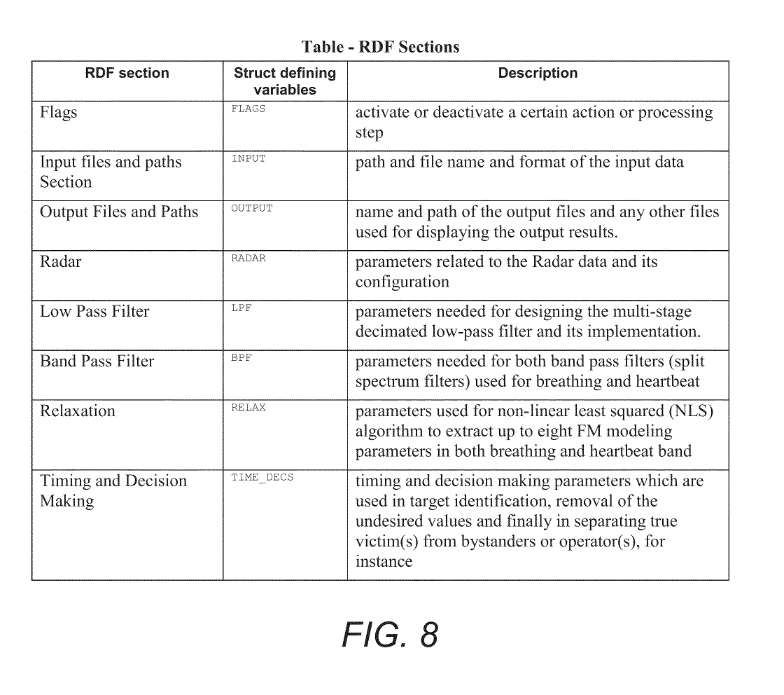

As described above, in some embodiments, the input parameters may be set in a text file called an RDF file. In some embodiments, the RDF file may consist of eight parts. An example of a sample RDF file with explanations for the various parameters is illustrated in FIG. 8. The struct name, (corresponding to structure in MATLAB) is used as a short abbreviation in the table. Although FIG. 8 illustrates an input file structured based on the MATLAB programming language, any of a variety of different input files may be specified for different programming languages as appropriate to the requirements of specific applications in accordance with embodiments of the invention.

In some embodiments, prior to start, all of the input parameters are read from the RDF file to configure the software. In particular, in some embodiments, a MATLAB script may read the RDF file and then a script may set the values so that they are accessible by all other scripts throughout the processing. Other implementations may use different programming languages and/or mechanisms to configure the parameters of the FINDER systems as appropriate to the requirements of specific applications.

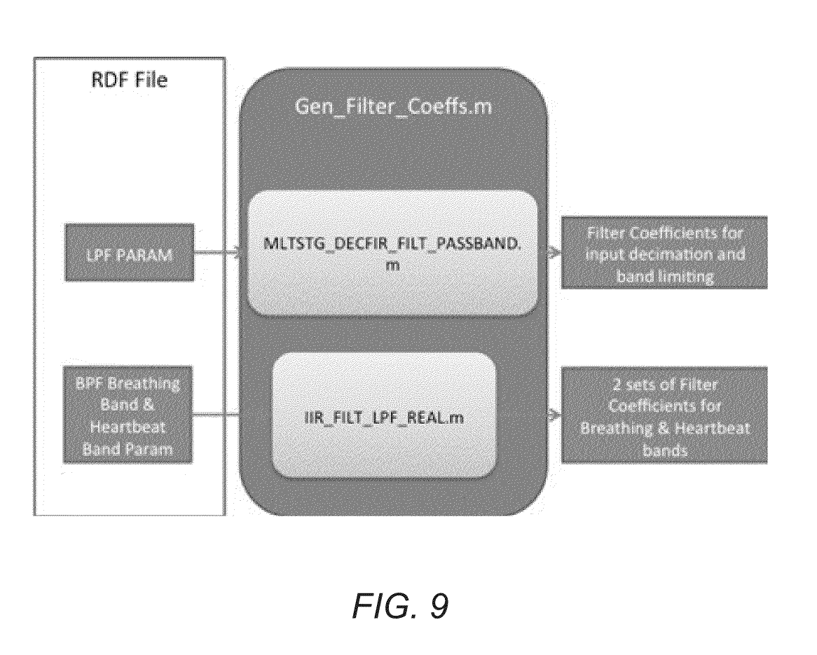

The values in sections LPF and BPF in the RDF file may be employed by a MATLAB script to generate the filter coefficients for multi-stage decimated low pass filter (LPF) applied to the time series of raw data as well as the coefficients used for band pass filtering (BPF) of the decimated LPF time series into two separate bands/time series, breathing and heart. The FIR and IIR coefficients may be generated by certain functions. In some cases, a RDF parameter allows selection of IIR or FIR filter configurations. In certain embodiments, the initial multi-stage decimation low pass filter uses a FIR filter and the band split processing uses IIR filter, as illustrated in the overall block diagram illustrated in FIG. 9. Other embodiments may use other filters and/or combinations of the FIR and IIR filters as appropriate to the requirements of specific applications.

Once the parameters are set and filter coefficients have been calculated, in some embodiments, the raw data for all available channels may be read by a script. The output may be a matrix containing all the time series data for all channels. Different embodiments may use different input file formats, including (but bit limited to) a MATLAB data file (.mat), binary file of floats, and an ASCII text file that is tab delimited.

### Per Channel Processing

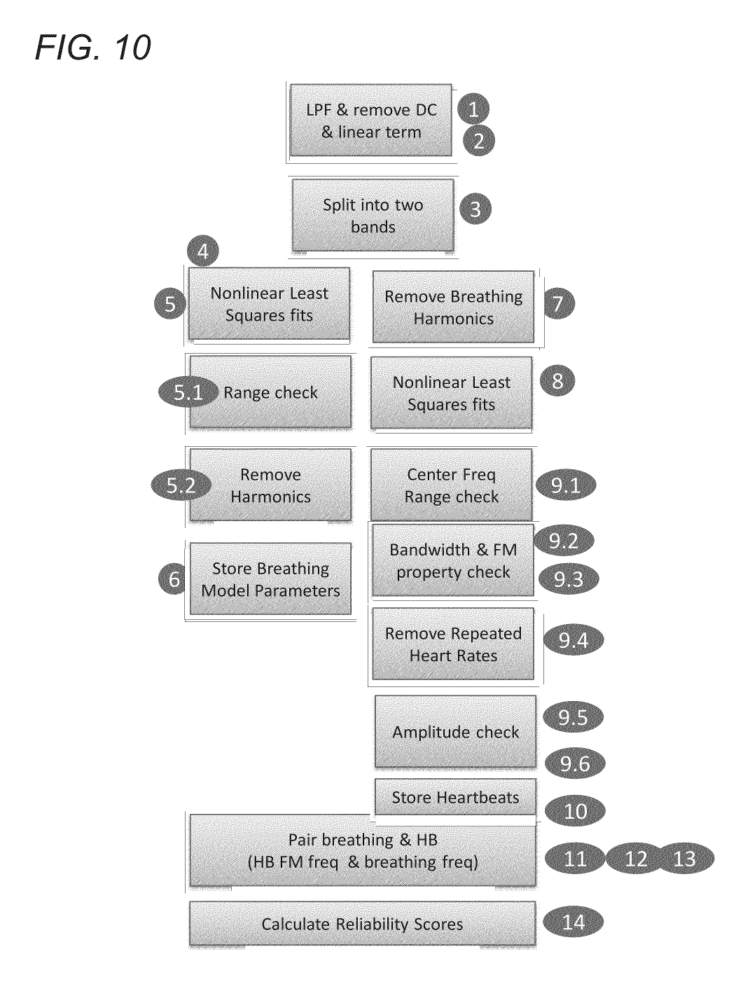

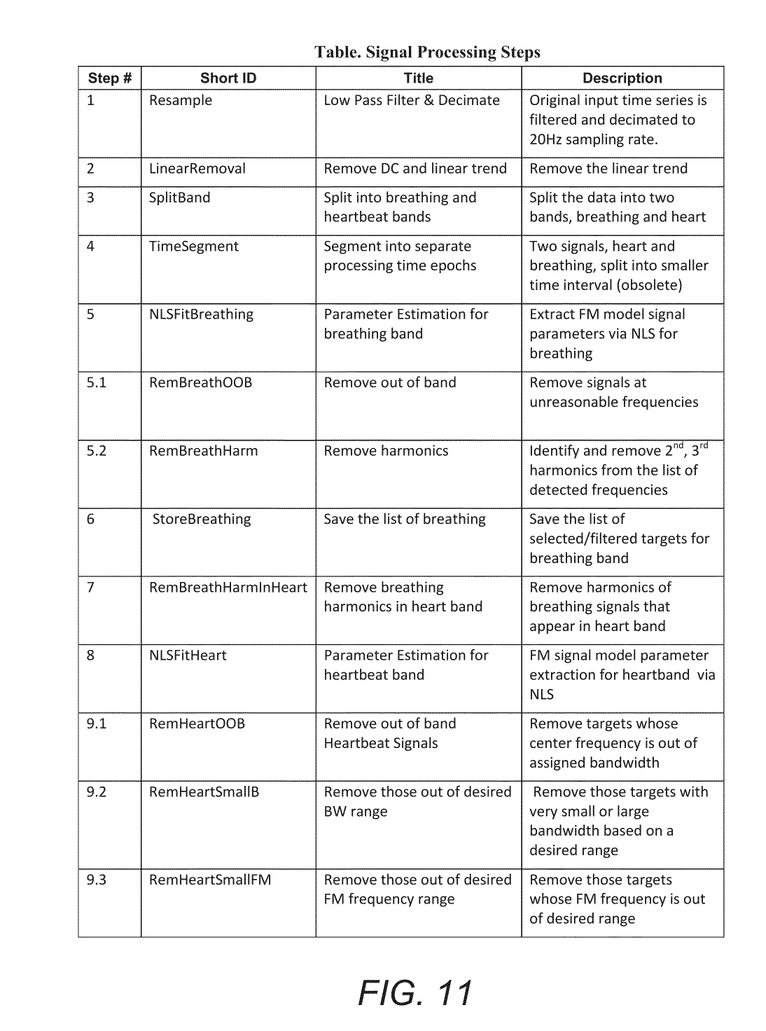

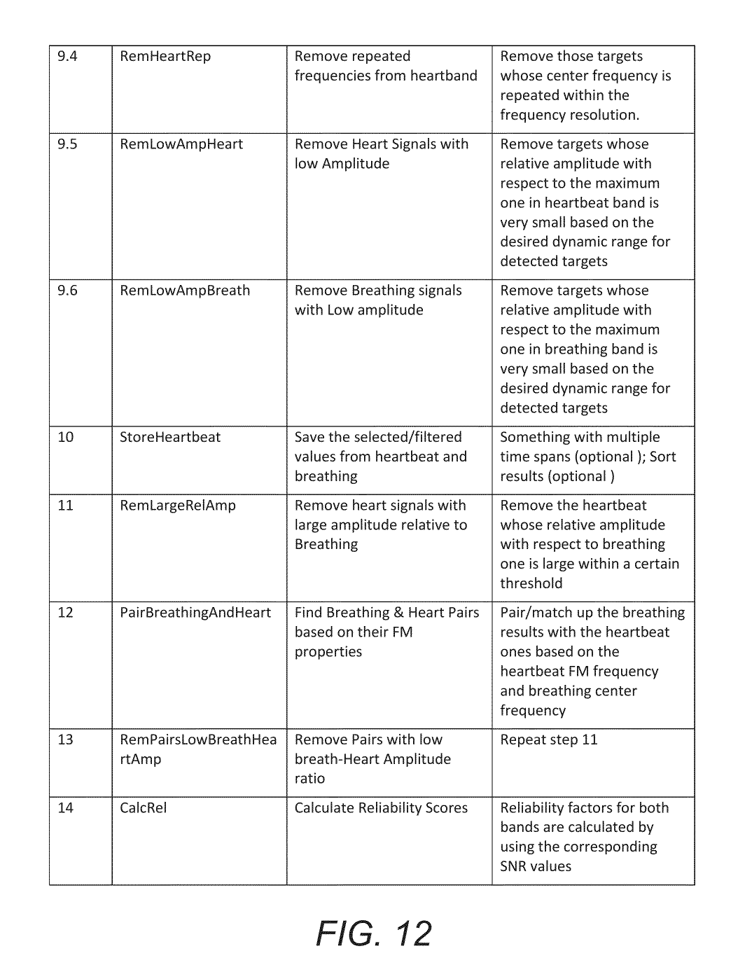

An example of a process for processing a channel signal in accordance with an embodiment of the invention is illustrated in FIG. 10. In some embodiments, the process is executed by a script. In other embodiments, the process may be executed by other mechanisms as appropriate to the requirements of specification applications. As illustrated, this process may be executed by each channel and include splitting the band into a heartbeat band and a breathing band. FIG. 11 and FIG. 12 illustrate a table that lists a step by step process of some embodiments for processing a signal. Although FIGS. 11-12 illustrate a series of steps, certain steps may be optional in certain embodiments and/or performed in a different order according to the requirements of specific applications in accordance with embodiments of the invention. A detailed description of each of the processing steps illustrated in FIG. 11-12 will now be described.

### 1. Low Pass Filter and Decimate

In some embodiments, the time series of a single channel (complex data, I(t)+jQ(t)) may be stored in a variable. In many embodiments, the mean value may be removed and subtracted from the same variable. The reason is that some desired frequencies are close to DC making it hard to see them and separate them when it comes to short time data collection. Prior to any filtering, there may be a few flags, for example specified in the RDF input file, to decide whether to process the amplitude, phase, or the complex data. In some embodiments, the signal model is a complex model based on the complex time series and therefore, uses the complex data.

As described above, in some embodiments, the zero-mean raw data may be decimated and low pass filtered into a new time series with a lower number of samples. In several embodiments, a series of Chebyshev FIR (finite impulse response) filters and corresponding decimators may be used. The group delay of the filter may be compensated by offsetting where the data starts prior to the decimation process. After filtering, a number of samples may be removed from the beginning of the sampled data because of the ringing initialization transient introduced by the FIR filter.

The FIR filter coefficients and group delay and the decimation factors may be generated by a function in the script. The corresponding parameters to implement such filter may be set in an LPF structure of the RDF file. In some embodiments, at the end, the residual mean of the band-limited data may be removed and subtracted from the same variable.

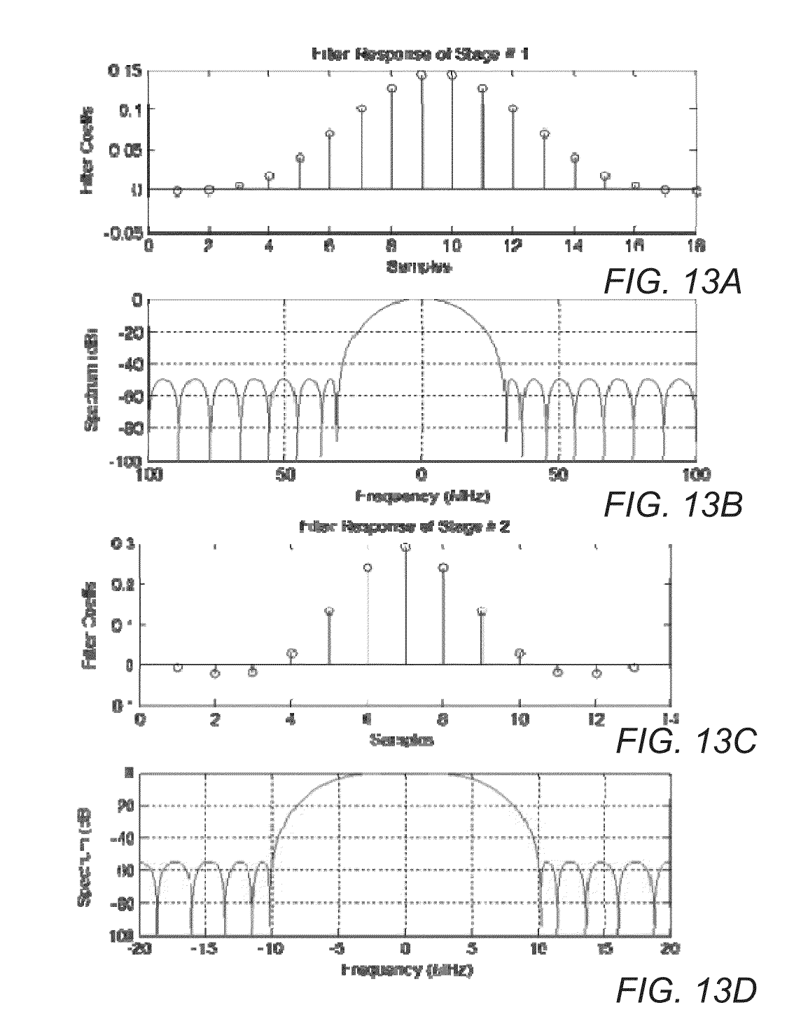

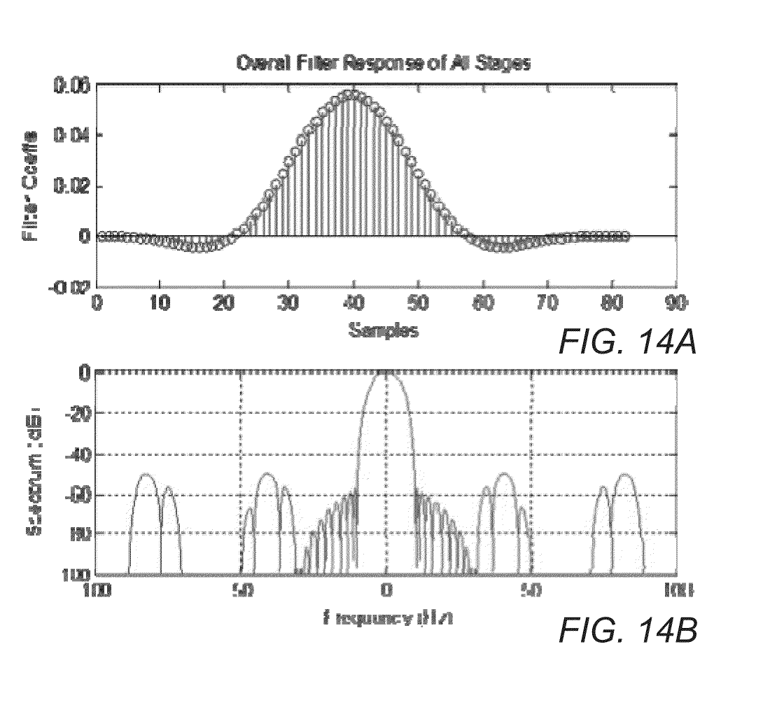

As an example, both the two stages as well as the overall filter response (both time domain and frequency domain) used for FINDER with input sampling rate 200 Hz and output sampling rate 20 Hz are shown in FIGS. 13A-D and FIG. 14A-B. In some embodiments, the signal after the stage one filtering may be decimated by a factor of five and after the second stage it may be decimated by a factor of two to bring down the sampling rate from, for example, 200 Hz to 20 Hz. As illustrated in this example, the pass-band width and stop-band width are 4 Hz and 20 Hz, respectively. Furthermore, the pass-band ripples and stop band attenuation are 0.1 dB and 50 dB, respectively. Although specific sampling rates and decimation factors are described above, any of a variety of sampling rates and/or decimation factors may be used as appropriate to the requirements of specific applications in order to process signals accordance with embodiments of the invention.

### 2. Remove DC and Linear Trend

In some embodiments, the linear trend using the linear least square fitting in the data may be removed. Moreover, any residual mean may also be removed. This may be necessary prior to BPF, which might use an IIR filter, and also prior to applying the non-least squared (NLS) process for feature extraction, all due to the possibility of the instability in filtering or weak convergence in recursive processing. In other words, in some embodiments, the data may need to be cleaned up prior to feature extraction.

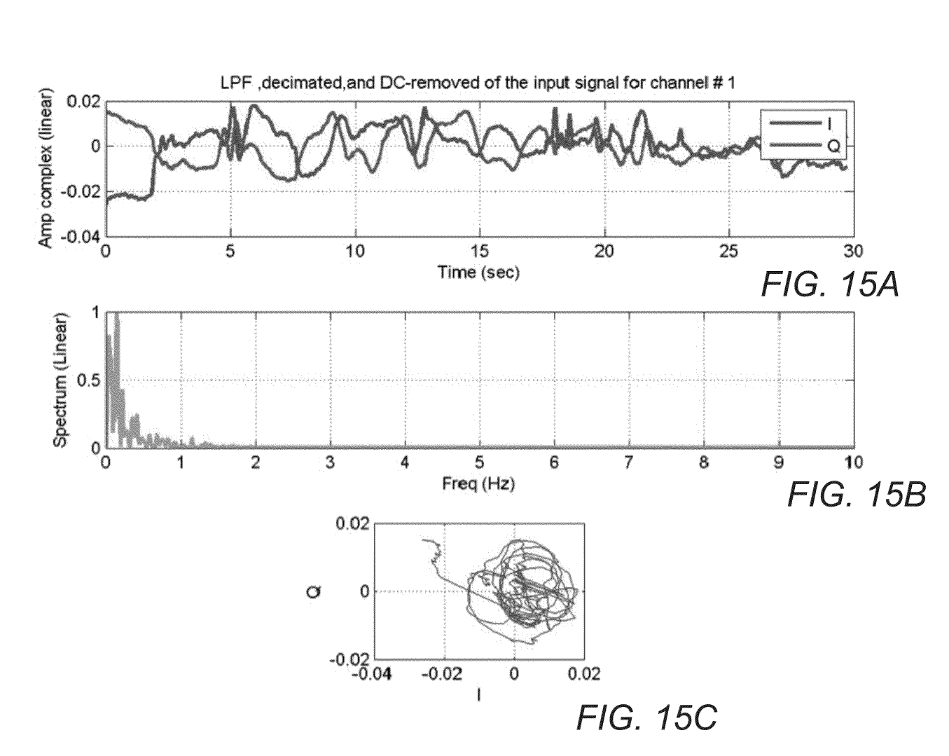

In some embodiments, an image, such as a .png image, may be generated from the cleaned data and may be used to carry the information for each channel or beam. The output of this stage may be stored in the same variable as input. An example image in accordance with some embodiments is shown in FIG. 15. Although FIG. 15 illustrates using an image to carry information for each channel, any of a variety of different mechanisms may be used to output information as appropriate based on the requirements of specific applications for outputting data in accordance with embodiments of the invention.

3. Split into Breathing and Heartbeat Bands

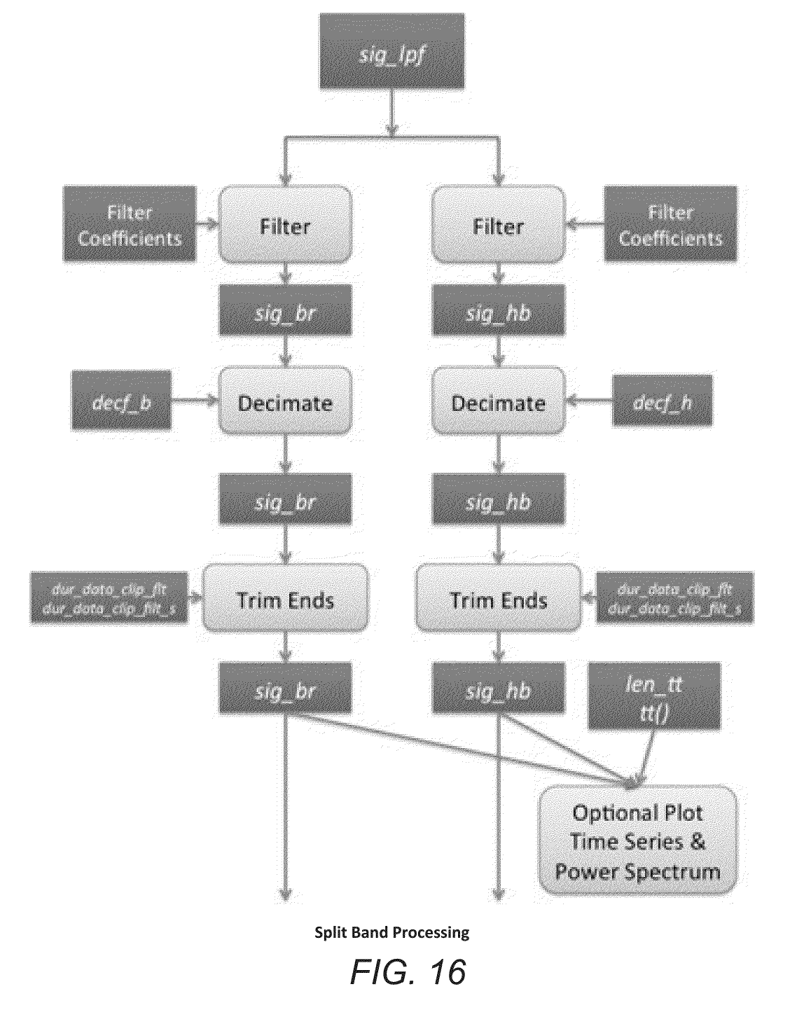

At this stage of the processing, in some embodiments, a single time series may be split into two time series/signals, breathing signal, and heart signal. Some embodiments may use two BPFs (split spectrum process plus residual mean removal for band-limited signals). Other embodiments may use other types of filters and/or mechanisms to split the time series signal into the separate bands. FIG. 16 illustrates an example of the data flow of this process, including the decimation to a lower sample rate and trimming of the ends of the time series. The filter coefficients may have been calculated before the input data file is read based on values from the RDF. The decimated sample rates for breathing and heartbeat may be identical, and calculated, along with the decimation ratios. In some embodiments, the amount of time to trim from the ends may also be determined.

In some embodiments, the decimation factor may be 1, (e.g., all at the same sample rate). In other embodiments, the decimation factor may be a different value, other than 1, based on the requirements of specific applications in accordance with embodiments of the invention.

In some embodiments, no trimming is done (e.g., the durations of data clip are both zero). In other embodiments, trimming may be done as appropriate to the requirements of specific applications.

In some embodiments, there may be two types of filters available in this processor: finite impulse response (“FIR”) and infinite impulse response FIR), and the selection may be set by corresponding flags in the RDF. Other embodiments may use other types of filters and or combinations of filters to process a signal.

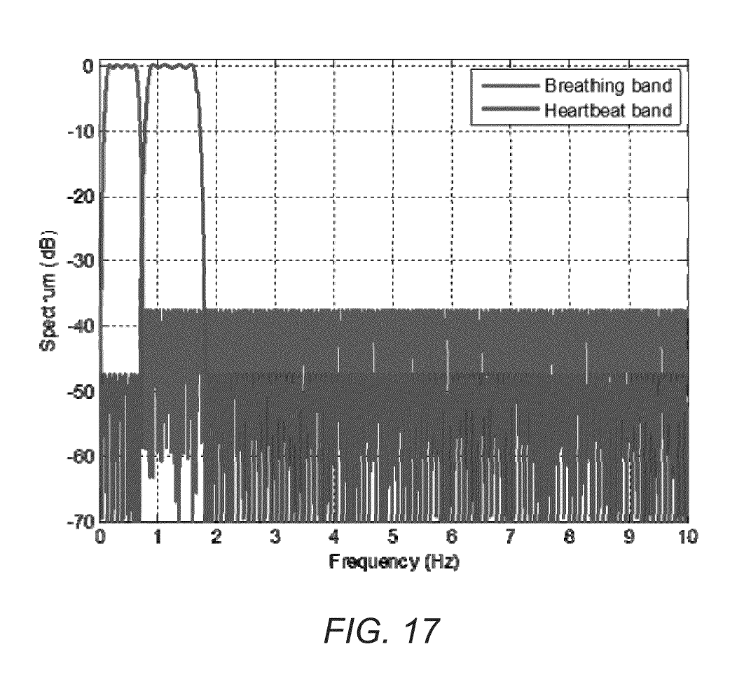

Similar to LPF, in some embodiments, the filter coefficients and group delays and decimation factors may be generated by the same scripts if FIR filter is selected. However, for the IIR case, the script may call a different function to generate the second order section digital filter for breathing/heartbeat and gain of the filter first, and then at convert into filter coefficients numerator of the transfer function and denominator of the transfer function of the filter). In some embodiments, the characteristics of the filters may be set in the RDF struct BPF. FIG. 17 shows an example of a sample of the bandpass characteristics for the two bands.

In some embodiments, due to the narrow bandwidth and fairly sharp transition (roll off) of the filters, the FIR filter length may be quite large compared to the length of the input time series samples. A large-length FIR filter may introduce ringing (a time-domain artifact) in the filtered samples and it also may have a larger group delay (more samples to initialize the filter). Therefore in some embodiments, IIR (infinite response response) may be preferred to FIR counterparts.

Unlike the FIR function which may generate single-side bandpass complex coefficients, the IIR function may generate real based-band coefficients. Therefore, for bandpass filtering, the input data may be first down-converted from a desired center frequency set by RDF file to a base band and then the data may be filtered and finally converted up to its initial center frequency.

Regardless of whether IIR or FIR filters are used, after filtering, in some embodiments, the data may be decimated and the ends may be trimmed at the beginning and at the end due to delays or artifacts. The decimation ratio and the amount of clips in time may be set in the RDF file. In some embodiments, the output of the split band filtering may be done in place and the results may be stored in variables.

In some embodiments, no decimation is done (the factor is 1) and no trimming is done (the durations are zero). Finally, in some embodiments, any residual mean may be removed and the results may be stored in the same variables. Although FIG. 16 illustrates a specific process for filtering and decimating a signal into separate bands, any of a variety of different processes that use different types of filters and/or decimation techniques may be utilized as appropriate to the requirements of specific applications for processing signals in accordance with embodiments of the invention.

4. Segment into Time Epochs For Processing

In some embodiments, the total time series may optionally be broken into a set of smaller time intervals with or without overlapping regions whose values may be set in the RDF file. In some embodiments, the interval may be set large enough compared to the total (e.g., 30 sec) data collection to avoid time division. The reason for that may be to reserve the maximum frequency resolution when time samples are converted into the frequency domain. For a longer data collection, some embodiments may use the time division by setting the processing time interval shorter than the total data collection time. This may, then, create a loop over several intervals, and the following may be applied to each channel, and may be repeated for each interval. At this stage, the number of time intervals may be determined and stored in a variable. The sampling frequency of time series at this stage after all those decimation and filtering may be stored in a variable which is used for both breathing and heartbeat since the same decimation factor may have been used for both bands defined in the RDF file.

The finest possible frequency resolution may be estimated based on the total length of the data in time (seconds) and stored in a variable. Then this value may be compared with any frequency resolutions set by the RDF file in order to make sure they are all bigger than this value and if not they may be replaced by this value.

### 5. Parameter Estimation for Breathing Band

In some embodiments, the NLS process may fit the complex input to a FM model with seven parameters as shown in equation (1B) below. The NLS process may be implemented by a function of the FINDER system. The output of this subroutine may include a number of detected targets that fit the model, their corresponding center frequencies (e.g. respiration frequency), complex amplitude, associated time and bandwidth values, FM index, FM frequency, FM phase, and the estimated noise power and the AutoRegressive (AR) model order used for SNR calculation plus a 6 dB bandwidth factor of the sinc function. The respective input parameters for this process may all be defined in a structure generated from parameters of the RDF file. Other embodiments may fit the input to other models with different parameters as appropriate to the requirements of specific application for processing signals in accordance with embodiments of the invention.

A relaxation process may be used to solve a non-linear least squared problem (NLS) for estimating the number of targets and their corresponding features based on a new proposed signal model. In some embodiments, a relaxation process from Ghaemi is used to solve the non-linear least squared problem. See Hirad Ghaemi, “Synthetic Aperture Weather Radar”, Master Thesis, Chalmers University of Technology, Goteborg, Sweden, 2008 available at http://elib.dlr.de/54507/1/MScThesis_H.Ghaemi.pdf . In some embodiments, the model used for FINDER may be an extended version of the one described in that reference. Other types of processes may be used to solve the non-linear least squared problem as appropriate to the requirements of specific application in accordance with embodiments of the invention.

In some embodiments, the complex signal model defined for FINDER may be called FM (frequency modulated) signal model (due to the FM term in the model) which may consist of seven parameters as given below in equation (1B) for K number of targets:

s(t)=Σₖ₌₁^{K}Dₖsinc(Bₖ(t−Tₖ))e^{i2πf}^{_{ck}}^{t}e^{ih}^{ₖ}^{cos(2πf}^{ₘₖ}^{t+φ}^{ₖ₎} (1B)

Where sinc is defined as

$$sin c ( x ) = \frac{sin ( π x )}{π x} \qquad ( 2 B )$$

The seven parameters for the k th target are complex amplitude Dₖ, the bandwidth Bₖ, the peak time Tₖ, the center frequency f_{ck}, the modulation index h, the FM frequency fₘₖ, and the FM phase φₖ. The objective may be to determine the number of targets (model order) fits the model with their seven respective parameters. Note that for heartbeat signals, in particular, this model may be a good representation of the Respiratory Sinus Arrhythmia (RSA) where the heart rate varies up and down with respiration.

Given the complex time series y(t) as an input, the input may be broken down into a signal term with the above-mentioned model plus an additive noise term v(t) (by noise meaning any undesired signal which doesn't fit the model). The fact that the system is linear, the noise term may be treated as an additive term.

Since the samples may be discrete samples, the time t may be replaced by discrete samples m with M total number of time samples (e.g., 600 for the FINDER with 30 sec data collection interval). These two terms may be related by the sampling frequencyf (e.g., 20 Hz for the FINDER) as:

m=fₛ·t, m=0, . . . , M−1 (3B)

In some embodiments, for simplicity, the sampling frequency fₛ term may be ignored and m may be considered to be an equivalent oft that is, m≡t , in the following equations.

Therefore, the time series input signal vector and the signal model vector, both with length M, may be expressed as follows

y(m)=s(m)+v(m), m=0, . . . , M−1 (4B)

s(m)=Σₖ₌₁^{K}Dₖsinc(Bₖ(m−Tₖ))e^{i2πf}^{_{ck}}^{m}e^{ih}^{ₖ}^{cos(2πf}^{ₘₖ}^{m+φ}^{ₖ₎} (5B)

As described above, the goal may be to estimate the seven unknown parameters of the model representing the feature of the target via minimization of the cost function C₁ given by non-linear squared (NLS).

C₁({Dₖ, Bₖ,Tₖ, f_{c}^{ₖ}, hₖ, fₘ^{ₖ}, φₖ}ₖ₌₁^{K})=∥y−GD∥² (6B)

Where

y=[y(0)y(1) . . . y(M−1)]^{T} (7B)

D=[D₁ D₂ . . . D_{K}]^{T} (8B)

G=[g(0)g(1) . . . g(M−1)]^{T} (9B)

g(m)=[g₁(m)g₂(m) . . . g_{K}(m)]^{T} (10B)

gₖ(m)=sinc(Bₖ(m−Tₖ))e^{i2πf}^{_{ck}}^{m}e^{ih}^{ₖ}^{cos(2πfm}^{ₖ}^{m+φ}^{ₖ₎} (11B)

T and ∥.∥ denote the transpose and Euclidean norm, respectively. Note that the bold letters may be employed for the vectors (one dimensional array).

K may be the estimated maximum number of targets which is determined automatically by the recursive process of the process using the Generalized Akaike Information Criterion (GAIC). See J. Li and P. Stoica, “Efficient mixed-spectrum estimation with application to target feature extraction.” IEEE Trans. Signal Processing, Vol. 44: pp. 281-295, February 1996.

Unlike the rest of the parameters, the cost function C₁ may be a linear function of the complex amplitudes Dₖ which can be readily determined by minimizing the linear least square of cost function. Thus,

$$\begin{matrix}{D}_{k} = \frac{{G}_{k}^{H} {y}_{k}}{{G}_{k}^{H} {G}_{k}} & ( 12 B ) \\ {y}_{k} = y - {\sum }_{j = 1 , j \neq k}^{K} {D}_{j} {g}_{j} & ( 13 B ) \\ {g}_{j} = {[ \begin{matrix}{g}_{j} ( 0 ) & {g}_{j} ( 1 ) & … & {g}_{j} ( M - 1 )\end{matrix} ]}^{T} & ( 14 B )\end{matrix}$$

H stands for Hermitian operation (complex conjugate transpose of a matrix or an array). Gₖ is the kth column of matrix G. A superior circumflex (“̂”) denotes an estimate of an actual parameter.

After inserting the equation (12B) into the equation (6B) and doing some mathematical manipulation, the minimization of C₁ may result in the maximization of the new non-linear cost function C₂ to estimate the six parameters, excluding the complex amplitude, as follows

$${C}_{2} ( {\{ {B}_{k} , {T}_{k} , {f}_{{c}_{k}} , {h}_{k} , {f}_{{m}_{k}} , {ϕ}_{k} \}}_{k = 1}^{K} ) = \frac{{\vert {G}_{k}^{H} {y}_{k} \vert}^{2}}{{G}_{k}^{H} {G}_{k}} \qquad ( 15 B )$$

The above maximization requires a six-dimensional search over six remainder parameters. To solve this, an alternating maximization procedure which updates one parameter estimate by fixing the rest may have been employed. Some embodiments may employ the procedure disclosed by Liu and Li, 1998. See Z .S. Liu and J. Ki., “Feature extraction of sar targets consisting of trihedral and dihedral corner reflectors,” IEE Proc. Radar, Sonar Navig., Vol. 145: pp 161-172, June 1998.

In some embodiments, to speed up the parameter estimation process for FINDER, the center frequency f_{ck} may be readily estimated by finding the dominant peak of the FFT (Fast Fourier Transform) of yₖ with enough zero padding (for better precision). The bandwidth Bₖ may also be estimated from the FFT of yₖ (e.g., 6-dB bandwidth around the peak, note that in FINDER the n-dB bandwidth may be determined and then scaled to 6-dB by using a 6dB scale factor so the bandwidth is the multiplication of the 6-dB factor and the estimated n-dB bandwidth with an arbitrary value n). If the bandwidth is set to 6-dB then the scale factor will be 1. The 6-dB factor is determined by the ratio of the estimated desired n-dB points and 6-dB points the sinc function).

The peak time Tₖ may be obtained by the Golden Section Search approach. Other embodiments may obtain the peak time using other approaches as appropriate to the requirements of specific applications.

The FM parameters may be simply determined via a three-dimensional search within the ranges defined in the RDF file.

Finally, the complex amplitude may be estimated by equation (12B).

Considering the above-mentioned strategy as well as the equations, the iteration steps of the relaxation process of some embodiments for solving NLS of equation 6B are:

Step (1): Assume K=1. Determine the first estimate of the seven parameters for the first target, i.e., {\hat{D}₁, \hat{B}₁, \hat{T}₁, \hat{f}_{c}^{₁}, ĥ₁, \hat{f}ₘ^{₁}, \hat{φ}₁} from y as described above.

Step (2): Assume K=2.

a) Compute y₂ with equation (13B) by using the estimated parameters obtained in step(1).

b) Estimate the second sets of parameters {\hat{D}₂, \hat{B}₂, \hat{T}₂, \hat{f}_{c}^{₂}, ĥ₂, \hat{f}ₘ^{₂}, \hat{φ}₂} for second target in the similar fashion.

c) Next, compute y₁ with equation (13B) by using the estimates {\hat{D}₂, \hat{B}₂, \hat{T}₂, \hat{f}_{c}^{₂}, ĥ₂, \hat{f}ₘ^{₂}, \hat{φ}₂} and then re-determine {\hat{D}₁, \hat{B}₁, \hat{T}₁, \hat{f}_{c}^{₁}, ĥ₁, \hat{f}ₘ^{₁}, \hat{φ}₁} from y₁

Repeat this iterative procedure at this step until the relative change in the cost function C₁ is smaller than a preset tolerance (e.g., 0.001 set for FINDER). In order to avoid time-consuming long iteration in case of weak and slow convergence, we limit the maximum number of iteration to be, for instance, 250 in FINDER.

Step (3): Assume K=3.

a) Calculate y₃ with equation (13B) by taking {\hat{D}ₖ, \hat{B}ₖ, \hat{T}ₖ, \hat{f}_{c}^{ₖ}, ĥₖ, \hat{f}ₘ^{ₖ}, \hat{φ}ₖ}, k=1,2 estimated at the end of the step (2).

b) Estimate {\hat{D}₃, \hat{B}₃, \hat{T}₃, \hat{f}_{c}^{₃}, ĥ₃, \hat{f}ₘ^{₃}, \hat{φ}₃} from y₃ as described in previous steps.

c) Next, compute y₁ by using {\hat{D}ₖ, \hat{B}ₖ, \hat{T}ₖ, \hat{f}_{c}^{ₖ}, ĥₖ, \hat{f}ₘ^{ₖ}, \hat{φ}ₖ}, k=2,3.

Iterate these three sub-steps until the relative change in C₁ is less than a desired tolerance.

Steps (K>3): Continue similarly until K is equal to K which is determined by minimizing the Generalized Akaike Information Criterion (GAIC) cost function in equation (16B). In other words, increase K until the GAIC cost function for the current iteration (let's say K+1) is bigger than that of previous one K.

Note that in FINDER, some embodiments also set another criterion to exit the iteration for K by putting a restriction on maximum acceptable number of targets (e.g., Kₘₐₓ=20) to avoid long iteration or infinite loop in case of large number of targets or bad input data.

GAIC(K)=M ln(∥e∥²)+4ln(M))(8K+1) (16)

e=y−Σₖ₌₁^{K}Dₖgₖ (17)

In some embodiments, the factor “8” in the above equation may be the total number of unknown parameters which are due to the fact that there “6” real parameters plus “1” complex parameter so the total real unknown parameters will be “8”. If any of the parameters in the model set to a fixed scalar then the total unknown parameters (real numbers) may be less than “8” and therefore a smaller value may be used in place of “8” in equation (16B).

In some embodiments, an autoregressive (AR) model is assumed for estimating the noise power used in estimation of SNR (as defined before, it's the ratio of the magnitude squared “D” in the signal model to the estimated noise power) and the reliability factor (defined as a function of SNR). After estimating the signal parameters for all detected signals and subtracting them from the input time-series data, the AR model parameters may be estimated from the autocovariance functions of the residual signal (after subtracting the estimated targets with their signal model from the input time series) by using the Yule-Walker equations. The order of the AR model may be determined based on the GAIC cost function defined in equation 2.13 of the “ Hirad Ghaemi, “Synthetic Aperture Weather Radar”, Master Thesis, Chalmers University of Technology, Goteborg, Sweden, 2008, p. 42-44 available at http://elib.dlr.de/54507/1/MScThesis_H.Ghaemi.pdf. The assumption here is after estimating the targets and subtracting them from the input time series through the FM signal model, the residual signal is stationary and ergodic Gaussian random process. The noise power may be finally determined after the AR model order and its parameters have been estimated via the Yule-Walker equation.

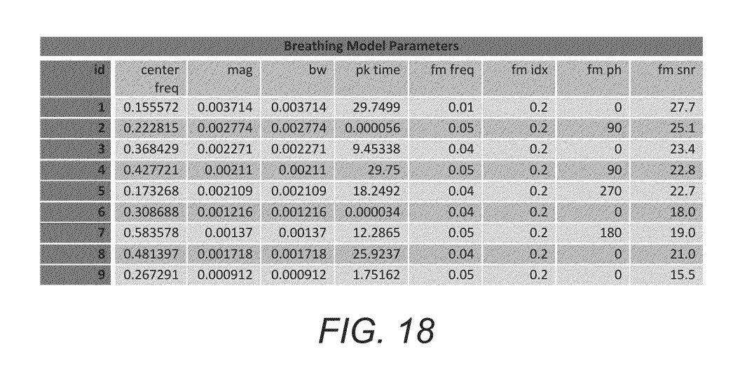

FIG. 18 illustrates a table that shows some sample data after the initial relaxation process performed on the breathing band data. As illustrated in FIG. 18, this sample data is preliminary and may need further cleanup before it can be used to identify actual victims.

In some embodiments, an initial cut may remove signals with frequencies that are outside the band limits defined in the RDF parameter (e.g., set to [0.04, 0.75] in this example) from the list of results if the corresponding flag is set to one. Even though the input to the relaxation process may be band limited, the process may find strong signals out of band (for instance, a strong motion appears out of band).

The remaining potential targets may go through a harmonic detection process to identify harmonics and then remove them from the list. Note that the results may be first sorted out in ascending order of the breathing center frequency prior to harmonic detection and removal. Then the repeated values may be removed from the list of breathing frequencies within a certain frequency resolution (e.g., 0.06 Hz), using RDF parameters, for example:
“Frequency resolution in decision and elimination process of breathing data (Hz)=0.06”
“Min Level below the carrier to considered as the second harmonic in harm cancel =0.0”
“Min Level below the carrier to considered as the third harmonic in harm cancel=6.0”

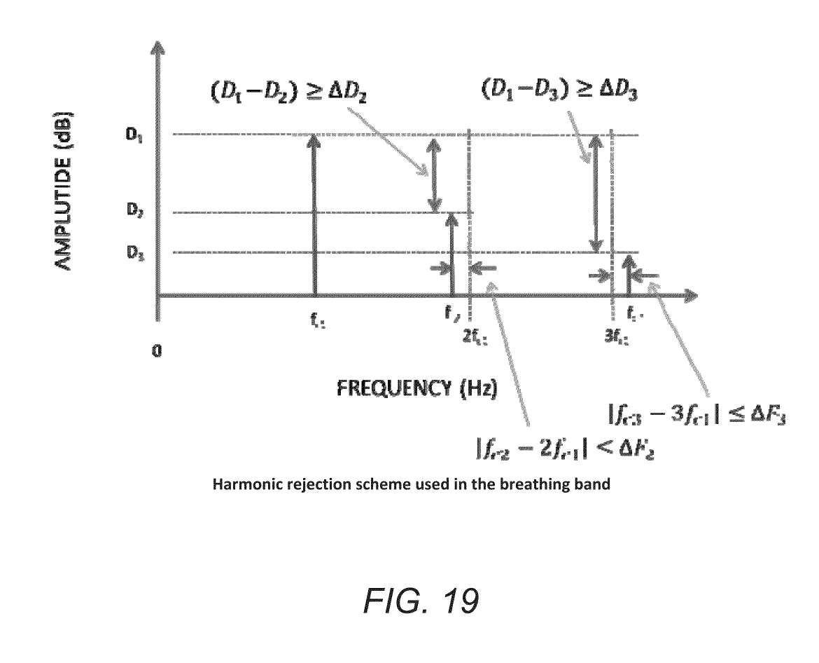

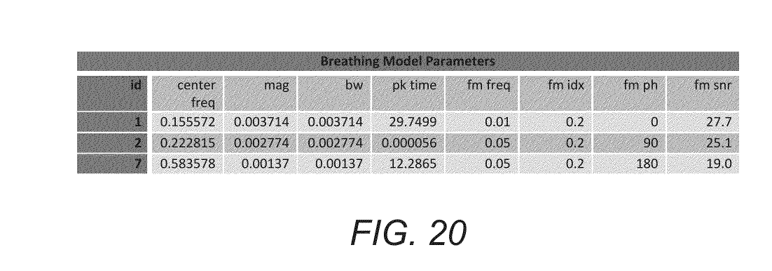

The harmonic rejection process may be based on relative amplitude ratio of the detected targets and their relative center frequency difference. An example of a harmonic rejection process in accordance with an embodiment of the invention is illustrated in FIG. 19. The amplitude thresholds ΔD₂ and ΔD₃ are for the second and third harmonics. The frequency resolutions for second and third harmonic ΔF₂ and ΔF₃are the same and set by a RDF parameter. The corresponding RDF parameters and their values as an example are shown above. An example of sample data after removal of the harmonics is illustrated in the table illustrated in FIG. 20. Although FIG. 19 illustrates a specific harmonic rejection process, any of a variety of different harmonic rejections processes may be used as appropriate to the requirements of specific applications in accordance with embodiments of the invention.

### 6. Store Breathing Values

In some embodiments, the process stores the selected values from the breathing list. Different embodiments may use different mechanisms to store the values as appropriate to the requirements of specific applications for storing values in accordance with embodiments of the invention.

### 7. Remove Breathing Harmonics in Heartbeat Band

In some embodiments, the process uses the stored breathing frequencies and performs harmonic rejection of the breathing frequencies on the heart band/signal up to a certain harmonic value via linear least square fit of complex CW (continuous wave) signals whose frequencies are all the estimated breathing frequencies as well as their desired harmonics set by the RDF file.

In some embodiments, the inputs to this step may be the breathing frequency list, number of harmonics, and the heartbeat signal (and its residual DC value may be removed). In some embodiments, the output may be a new time series of the heartbeat signal. Other embodiments may include a different set of inputs and/or outputs as appropriate to the requirements of specific applications.

### 8. Parameter Estimation for Heartbeat Band

In some embodiments, the process for parameter estimation may be repeated for heart signal (heart band) with certain parameters defined in, for example, an RDF file. As an example, these parameters are:
“FM frequency range used for heartbeat signal model (start,stop,step) (Hz,Hz,Hz)=0.01,0.35,0.01”
“FM index range used for heartbeat signal model (start,stop,step)=1.0,1.05,0.05”
“FM initial phase range used for heartbeat signal model (start,stop,step) (deg,deg,deg)=0,270,90”

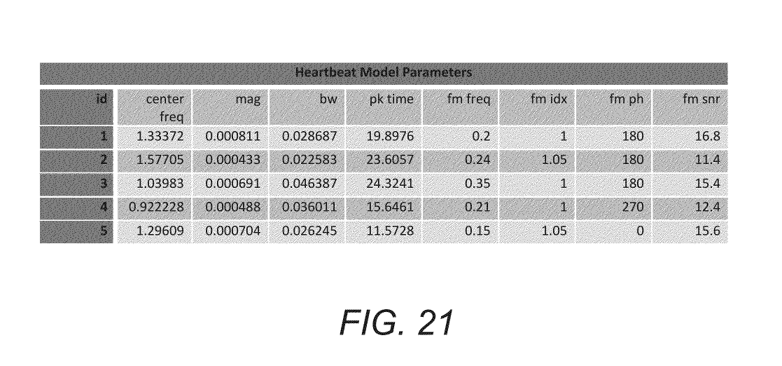

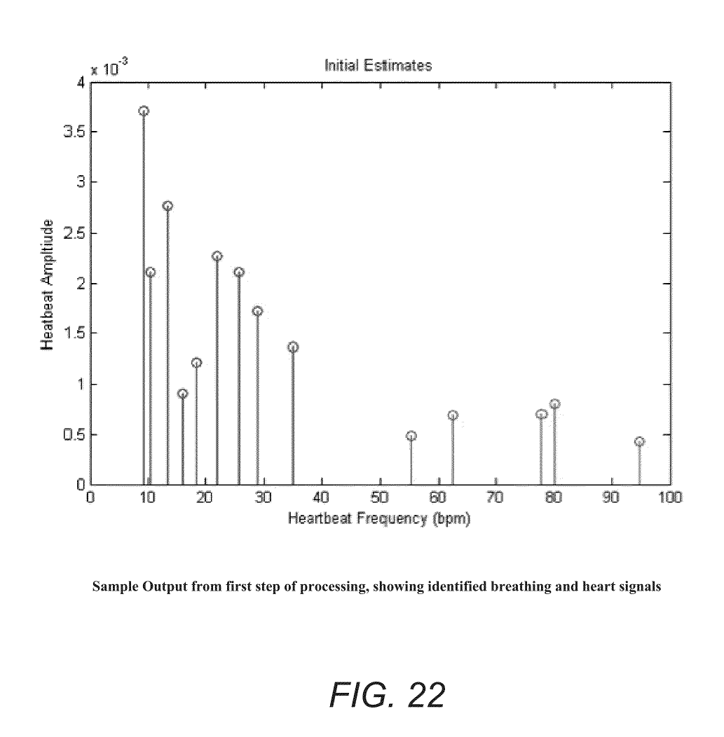

An example of a set of initial estimated values are listed in the table illustrated in FIG. 21. FIG. 22 illustrates an example of the initial estimated values for both breathing and heartbeat band. Other embodiments may use a different set of parameter values and/or different types of input files as appropriate to the requirements of specific applications for performing parameter estimation in accordance with embodiments of the invention.

### 9. Remove Unreasonable Heartbeat Signals

In some embodiments, once a list of targets with their corresponding FM parameters is obtained, the elimination process of undesired ones may start with those whose heartbeat frequencies are out of the heartbeat band as set by, for example, the RDF file.

In some embodiments, those targets with bandwidth and FM frequencies out of defined range may be removed. In some embodiments, the typical RDF file entries for bandwidth and modulation frequency, respectively, are:
“The smallest and largest acceptable bandwidth for both breathing and heartbeat (Hz, Hz)=0.0,0.13”
“Threshold for FM frequency in removing detected heartbeats with corresponding small values (Hz)=0.02”

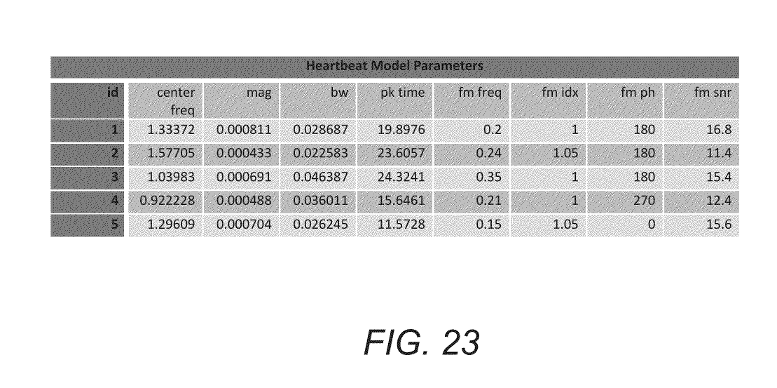

An example of the corresponding list of values after this elimination process may be as illustrated in the table in FIG. 23.

In some embodiments, the process may include an optional step for removing the intermodulation component of breathing in heartbeat band. Other embodiments may keep the intermodulation component as appropriate to the requirements of specific applications.

In some embodiments, the repeated frequencies with smaller amplitude may be removed within the frequency resolution set by the RDF file.

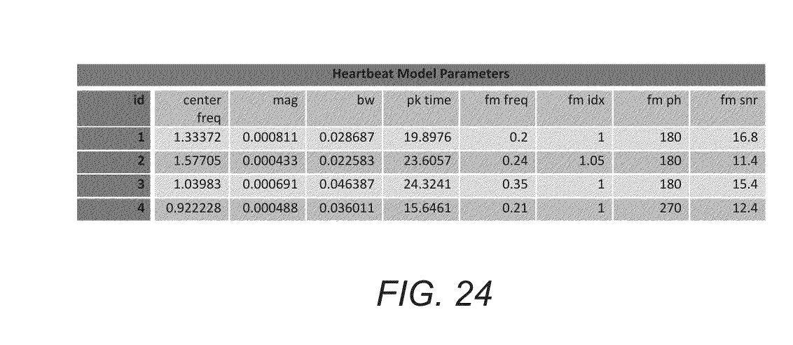

An example of results after this selection process is shown in the table illustrated in FIG. 24.

Finally, the processor may remove those targets (both for breathing band and for heartbeat band) with relatively very small estimated amplitudes as defined by an RDF parameter.

For example, an RDF parameter may specify a relative threshold below the max used for small target detection and removal (dB, positive)=25.0.

In some embodiments, the relative values may be defined with respect to the max detected amplitude (maximum estimated parameter D in dB), separately, for each band or signal. Other embodiments may specify the relative values with respect to other characteristics as appropriate to the requirements of specific applications.

In some embodiments, the inputs and outputs may be seven FM parameters, “D_h”, “F_h”, “B_h”, “T_h”, “fm_h”, “h_h”, “p_h”, and the corresponding SNR “snr_h” throughout the selection process. Other embodiments may use other inputs and/or outputs for the FM parameters as appropriate to the requirements of specific applications.

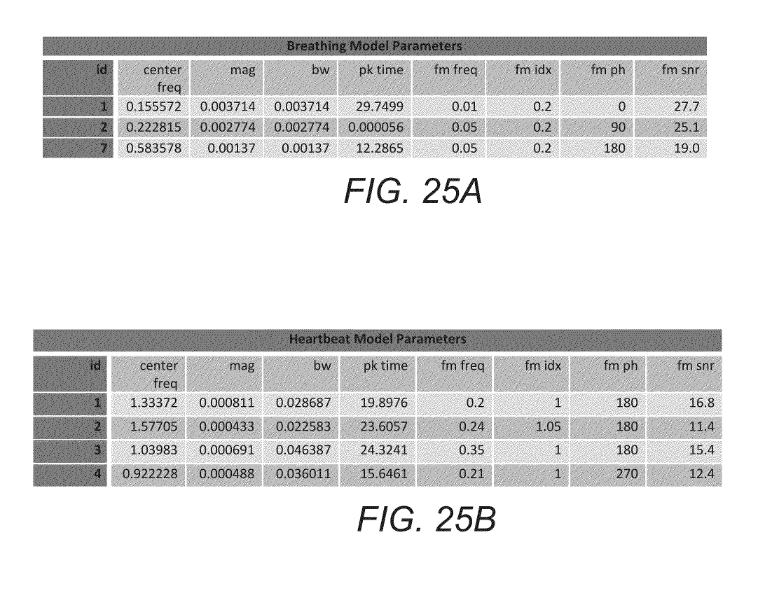

An example of the corresponding results for both bands after this selection process is illustrated in the table in FIG. 25A for the breathing model parameters and FIG. 25B for the heartbeat model parameters.

### 10. Store Heartbeat Values

Some embodiments may store the selected values of heartbeat band (e.g., all seven parameters in the FM signal model) after the above-mentioned filtering/selection process. In embodiments that use one time interval for FINDER, the process may simply sort out the results for both bands in the descending order of magnitude (absolute value of parameter “D” in signal model). In embodiments that use time division, the process may only take the minimum number of pairs (equal number of breathing and heartbeat) which occurs most frequently among all the intervals. Other embodiments may output the values using different mechanisms as appropriate to the requirements of specific applications in accordance with embodiments of the invention.

Note that for the time division, in order to properly choose the right results among several ones may be based on some certain criteria, for instance, their statistics of occurrence, their relative signal power, their SNR, the range of acceptable values and so forth.

In some embodiments of the FINDER system may use a short (30 sec) data capture and may process the whole interval for the sake of the speed. Other embodiments may use a different and/or longer duration as appropriate to the requirements of specific applications.

It's noteworthy that the thirty second time interval used for FINDER may be assumed to be the shortest interval for the single interval processing. For this data interval or shorter data take, the time division processing may not be recommended.

11. Remove Heartbeat Signals with Relatively Large Amplitude

At this stage, the lists from heart signal may be filtered in terms of their relative amplitudes prior to pairing them up. All amplitudes from the heartbeat may be compared to the max amplitude from the breathing list. The max heartbeat amplitude should be below a threshold set by an RDF parameter, such as “the min ratio of breathing amp and its heartbeat used in false target removal (dB, positive)=11.5”. Otherwise, that target may be removed from the heart list. The inputs and outputs may be the seven parameters of the FM model as well as the respective SNR of the heart signal.

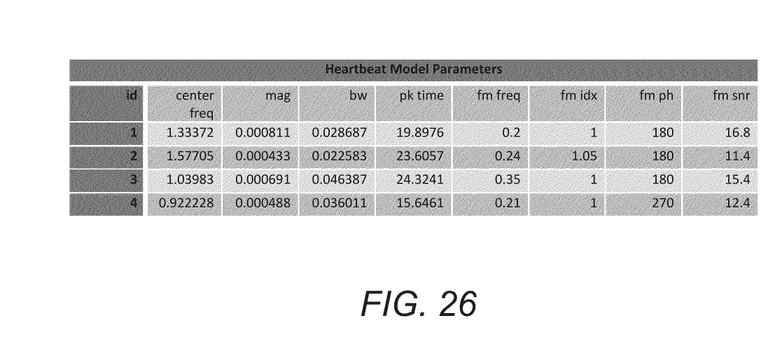

An example of remaining heartbeat values after this process is provided in the table illustrated in FIG. 26. Note that in some embodiments this elimination process may be repeated after pairing the breathing and heartbeat results. Other embodiments may apply a different process after pairing based on the requirements of specific applications.

### 12. Find Breathing & Heartbeat Pairs

In some embodiments, the breathing and heart values may be paired up based on the difference between breathing frequency f_{c}^{b} and the FM frequency of the heart g shown in equation (2C) below. Some embodiments first may find the pairs which minimizes the difference |f_{c}^{b}−fₘ^{h}|) and then check the difference to make sure they are less than a threshold defined by an RDF parameter called, for example, “the maximum abs difference between breathing freq and FM freq of heartbeat in a matched pair to be a true target (Hz)=0.5”. This threshold is shown by Δ_{bh} in the equation below. The rest of results which don't have a pair may be removed from the list. At this point, the pairs of results may consist of both respiration as well as heart rates.

|f_{c}^{b}−fₘ^{h}|≦Δ_{bh} (2C)

In some embodiments, this may be the first way to identify human target versus artifacts or undesired targets. This value may be estimated through a calibration process.

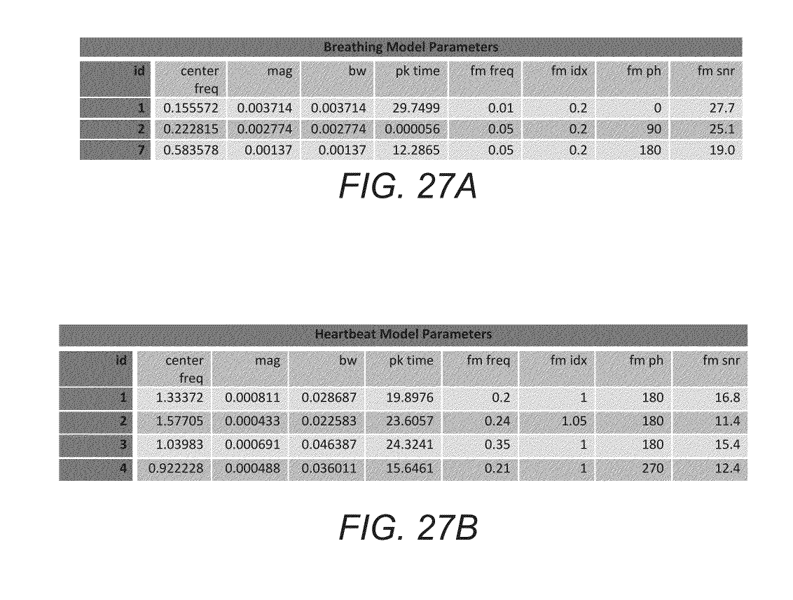

The inputs and outputs may be the seven parameters of FM model for both breathing and heartbeat. An example of parameters of the FM model for the breathing band is illustrated in FIG. 27A and the heartbeat band in FIG. 27B. Although FIG. 27A and FIG. 27B provide an example set of values for the model parameters, any of a variety of different values may be used as appropriate to the requirements of specific applications in accordance with embodiments of the invention.

13. Remove Pairs with Low Breath-Heartbeat Amplitude

In some embodiments, the process may reapply the step 11 on each pair separately to remove those pairs which don't have minimum amplitude ratio between breathing and the respective heartbeat in a pair. However, the process may compare the amplitudes within a pair, (rather than against the maximum breathing amplitude as in step 11). That is

$$10 \cdot {log}_{10} \frac{{D}_{b}}{{D}_{h}} \geq {A}_{t} \qquad ( 3 C )$$

The threshold Aₜ may be set by an RDF parameter.

For example, the min ratio of breathing amp and its heartbeat used in false target removal (dB,positive)=7.5

In some embodiments, this is the second way to eliminate non-human targets from the list. The corresponding RDF value may be determined experimentally through a calibration process.

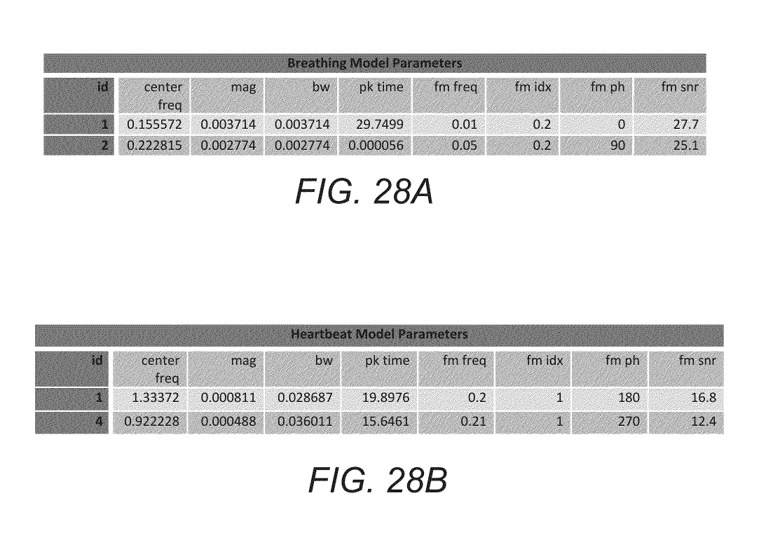

An example of the corresponding results for both breathing and heartbeat after this elimination process may be tabulated as illustrated by the table in FIG. 28A for the breathing model parameters and FIG. 28B for the heartbeat model parameters.

### 14. Calculate Reliability Scores

Some embodiments may report the frequency values, breathing rate and heartbeat rate, with their respective reliability, REL, in percentage, factors calculated using the estimated SNR values in linear scale as follows:

$$REL = 100 * ( 1 - \frac{1}{\sqrt{1 + SNR}} ) \qquad ( 4 C )$$

For example, in the log file, the reliability factors that are not reported in percentage as follows:
Breathing Reliability factors: 0.958643 ,0.944662 (unitless)
Heartbeat Reliability factors: 0.857681 ,0.767805 (unitless)

Some embodiments also report a joint reliability factor(s), ReI_{BH}, in the log file which is the geometric mean of the reliability factors of heartbeat and breathing pair. That is

Rel_{BH}=\sqrt{Rel_{B}·Rel_{H}} (5C)

### Wrap Up per Channel Processing

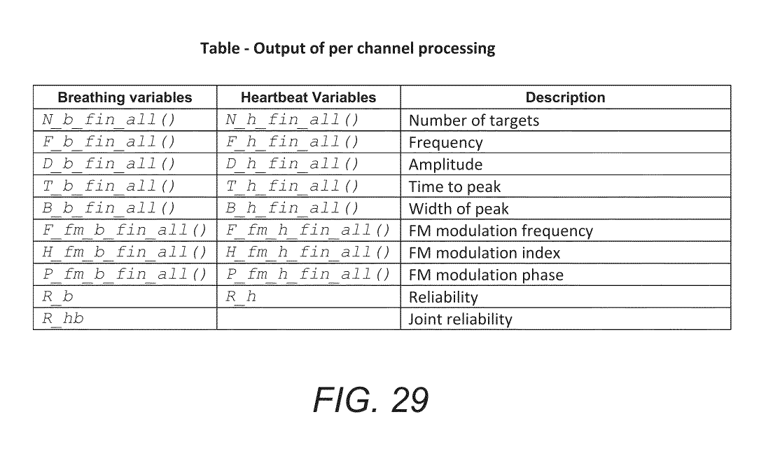

At this point, human targets (either as victim, bystander, or operator) may be identified with their respective parameters for each channel or beam. An example of variables that may be used to output information in accordance with embodiments of the invention is illustrated by the table in FIG. 29. In some embodiments, the individual channel values may be stored into a larger array. Other embodiments may use different mechanisms to store channel values as appropriate to the requirements of specific applications.

### Multichannel Processing

In some embodiments, after having all the results from all beams, the multi-beam processing may be performed to decide whether the targets detected in the front beam is true victim(s) or just bystander(s) or operator(s) to some extent. Multi-beam processing may start after all parameters are estimated and listed from all channels/beams described above. In some embodiments, there are two approaches used for multichannel processing, referred to here as “type 1” and type 2″ for convenience. In some embodiments, the following names have been used for the two types of processing:

Type 1—Signal Comparison

Type 2—Feature List Matching

The type 1, Signal Comparison, approach may take signals that have been detected in the front beam and see if they occur in the other beams at higher amplitudes. In particular, a target to the side of the radar will likely have a stronger return than one in front via a reflected path. The actual antenna patterns for each beam may be the product of the transmit beam and the individual receive beams, so they are not symmetrically disposed around the FINDER system.

The type 2, Feature List Matching, approach may compare the result sets from each beam and look for matches in the parameters, without trying to actually fit the originally acquired data.

The results may be reported as well as the number of true targets and number of bystander(s)/operator(s). In some embodiments, the actual processing may be split out into two separate processes, such as different MATLAB scripts.

### Type 1 Multibeam Processing—Signal Comparison

In some embodiments, a first script calls a function and the inputs may be the FM parameters of the front beam and the BPF filtered data of all channels/beams for breathing and heartbeat obtained after the split spectrum stage. The FM fitting process may look for signals that are listed as being in the front beam also occurring in the other channels, and if they are stronger in the side or back, it may declare them as spurious or false targets.

In some embodiments, the output may be the id/index of the false targets in the list of targets of front channel/beam. Some embodiments use the FM parameters excluding the amplitude of the list of detected targets in the front beam (pairs with both breathing and heartbeat values) and try to fit them to the filtered data (after split spectrum) of other channels/beams (from side and back beams) and then compare their estimated amplitude (power) obtained via linear least squared fit with the detected ones in the front beam. If they appear stronger in the side or back beam for the pair (breathing and heartbeat) then that target may be eliminated from the victim list in the front channel. In some embodiments, there is a comparison threshold that sets the relative level used for comparison, and changing that level may have the effect of widening or narrowing the effective “detection area” in front of the radar. This threshold in an RDF file may be, for example, “Multi beam comparison level relative to channel one used in FM Fitting (dB)=0.0” with value 0 dB.

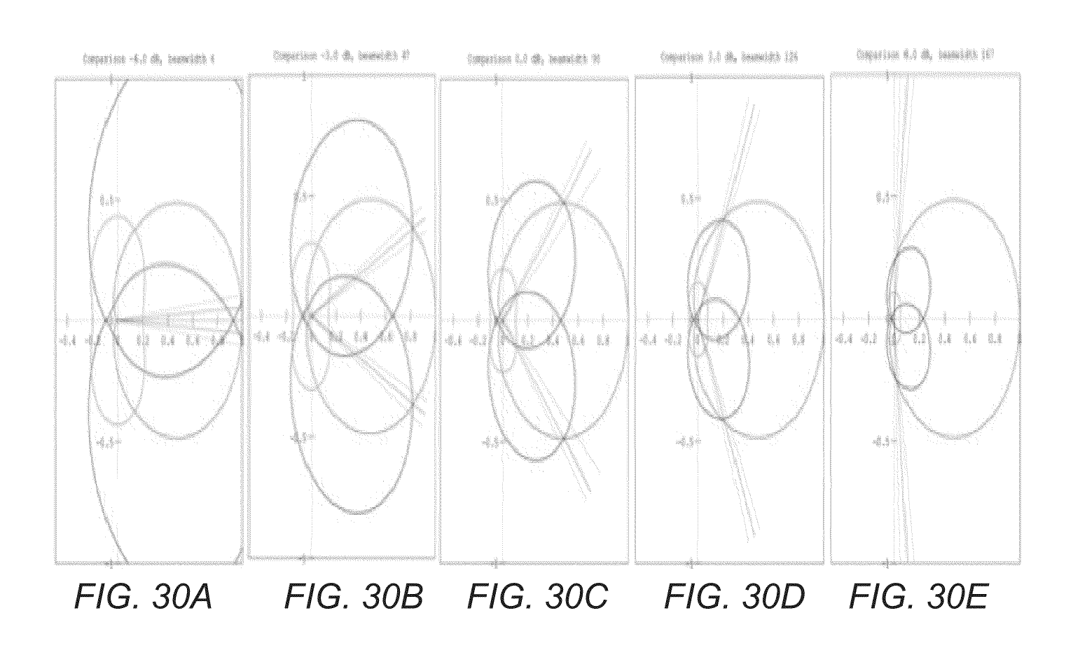

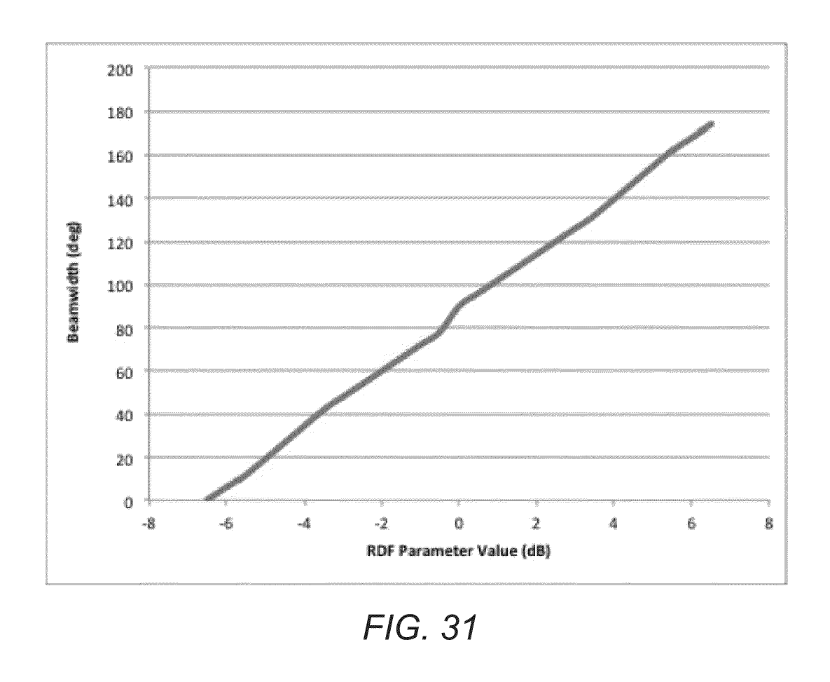

FIGS. 30A-E illustrate an example of the effective bystander rejection azimuth range or beam width for various comparison thresholds from −6 dB to +6 dB. For example, a default value of 0 dB may provide a beam width of approximately 90 degrees. In realistic circumstances, the patterns may not be as smooth, nor is the propagation from the victim uniform. In fact, with the monochromatic, single frequency illumination, there could be local intensity variations, so the dotted lines show the change in the decision edge for a 0.5 dB change in relative amplitudes. FIG.31 illustrates an example of the approximate “beam width” as a function of an RDF parameter.

In some embodiments, the FM fitting of breathing may use a set of fixed values for some of the parameters of front beam instead of estimates. The reason is to fit the values of critical parameters from front beam. An underlying assumption here is that the breathing signal is a pure unmodulated sinusoid (the matching is done on the fundamental, so any harmonic content doesn't enter into the fit) so the process may use zero values for the FM parameters. This may avoid any error in the estimation of the FM parameters (FM index, FM frequency, and FM phase) of the signal model. Nevertheless, some embodiments may include the FM parameters for breathing signal from the front channel in the FM fitting of multi-beam processing. The final results shouldn't change as these values are pretty small for a breathing signal.

In some embodiments, the reference time is also set to zero: essentially, this is equivalent to the assumption that the magnitude of the breathing signal is essentially constant over the, for example, 30 second fitting interval.

On the other hand, in some embodiments, for the heartbeat, the process sets the FM index to one and the FM phase to zero to make sure it's an FM signal with a fixed FM index. In some embodiments, the process may adjust the FM index to match the variation in heart rate (RSA) as a function of breathing, but for most humans, the amplitude of the RSA is roughly constant at low respiration rates.

The decision for the false target may be made based on the fact that both false target index array for breathing and heartbeat are not an empty array. Therefore, the detected target in the front beam may in fact be a bystander.

Some embodiments use a MATLAB function for FM fitting. This function employs the well-known closed form expression for the solution to the linear least square fit problem to calculate the corresponding complex amplitudes, A_{c}, for all channels in the FM signal model s(t), using the complex input 2-D array y₁ (contains the time series for all channels) as follows:

s(t)=sinc(B₁(t−T))e^{i2πf}^{_{c}}^{t}e^{ih cos(2πf}^{ₘ}^{t+φ)} (6C)

Where

B₁=α_{6 dB}×B (7C)

The linear least squared solution is given as

$${A}_{c} = \frac{{s}^{H} {y}_{1}}{{s}^{H} s} \qquad ( 8 C )$$

The 6-dB factor α_{6 dB} is used to calculate the correct 6 dB bandwidth of the sinc function in the amplitude (not power) signal model explained above.

Therefore, the inputs to this function is time series of data for all channel in 2-D array as well as the desired FM parameters. The output may be an array of complex amplitudes for four channels stored in variables for breathing and heartbeat, respectively.

Once the complex amplitudes for all beams being calculated, their absolute values may be converted into dB and then compared the amplitude from front beam to that of rest of channels for both breathing and heartbeat values separately, within a magnitude tolerance (in dB) set by RDF parameter (ΔA in equation (9C)):

“Multi beam comparison level relative to channel one used in FM Fitting (dB)=0.0”

In order for a target in front to be considered a false target, both breathing and heartbeat magnitudes of one of the other channels (Aᵢ^{b} and Aⁱ^{h} for i=2,3,4) may be bigger than those of the front channel(A₁^{b} and A₁^{h}) within a desired threshold/tolerance ΔA. That is

{Aᵢ^{b}≧A₁^{b}+ΔA(dB) and Aᵢ^{h}≧A₁^{h}+ΔA(dB)} i=2,3,4 (9C)

Type 2 multibeam processing—Feature List Matching

In some embodiments, the type 2 multibeam processing script may look at the final FM model parameter sets found in all beams. In some embodiments, the inputs may include the breathing and heartbeat rates stored in variables with their corresponding reliability factors generated by the main script respectively. The output may be again an array of indices for the list of false target in the front beam.

In some embodiments, first, both heartbeat and the breathing frequency of a pair from the front channel may be compared with pairs from other channels.

If both heartbeat and breathing frequencies from the front channel (e.g., channel 1) are close to those from either of the other channels within the frequency tolerance set by RDF parameters stated below, the process may proceed with the second step.
“Frequency resolution in decision and elimination process of breathing data (Hz)=0.06”
“Frequency resolution in decision and elimination process of heartbeat data (Hz)=0.06”

In some embodiments, the first step can be depicted in a simple equation as follows:

{Relᵢ^{b}<Rel₁^{b}ΔR(%) or Relᵢ^{h}>Rel₁^{h}+ΔR(%)} i=2,3,4 (10C)

The ΔF^{b} and ΔF^{h} are the frequency tolerances for breathing and heartbeat, respectively.

Second, if either of the reliability factors of breathing or heartbeat from channels the side and back channels are bigger than that of front channel within the tolerance set by, for example, the following RDF parameter, that target may be considered as a false target.

“Reliability difference used for victim and bystander decision (%, positive)=5”

This step can be shown in an equation as follows:

{ Relᵢ^{b}>Rel₁^{b}+ΔR(%) or Relᵢ^{h}>Rel₁^{h}+ΔR(%)} i=2,3,4 (11C)

The parameter ΔR is the tolerance of the reliability set by RDF file. In some embodiments, it is the same for both breathing and heartbeat. In other embodiments, it may be different for different bands.

In summary, if both conditions above are valid then that respective target in the front beam may be a false target (considered to be a bystander or an operator rather than a true victim).

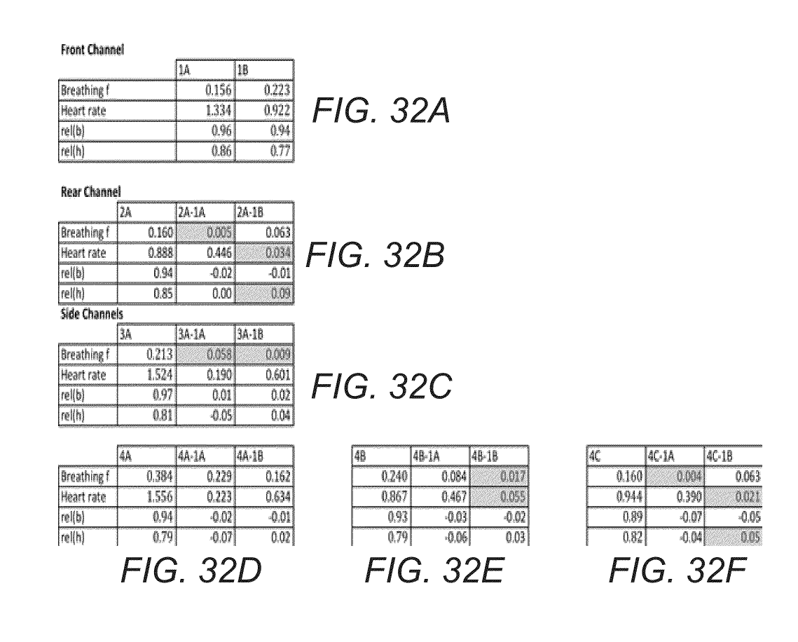

As an example, FIGS. 32A-F show the detected targets at each beam prior to multi-beam processing. In this example, the frequency resolution for both breathing and heart rates are about 3.9 bpm (set by the RDF file to 0.065 Hz=3.9 min⁻¹). In this example, the values reported in the log file are in Hz but those in the html files are in bpm (beat per minute, min⁻¹). The conversion is:

bpm=60×Hz (12C)

The pair in the second row of the front beam (heart=55 bpm and Resp=13 bpm) seems to have close match within +/−3.9 bpm, for both heartbeat and breathing rates, at the second row of the side beam (beam #4) and that of back beam (back beam #2). Since close matches have been found with the respect to the breathing and heartbeat rates, the respective reliabilities may need to be checked prior to final decision.

In the example, the corresponding reliabilities for the breathing in beam #2 and beam #4 are 94% and 93%, respectively, which are equal or less than that of the beam #1 , 94%, within the tolerance 5%. In other words, they are less than 99% (94%+5%). Therefore, the breathing doesn't meet the requirement to make this pair of the front beam a false target. However, the corresponding heartbeat pair appears larger (within 5% threshold) in beam #2 (85%) than in the beam #1 (77%). The 85% of beam #2 is bigger than 82% (77% +5%).

Since, one of the pair (heartbeat in this case) does pass the test for false target identification, the second row of the results of the front beam (beam #1) is considered a bystander or operator rather than a true victim. Therefore, in the final result (true victim list), only the results of the first row of the front beam is reported as true victim.

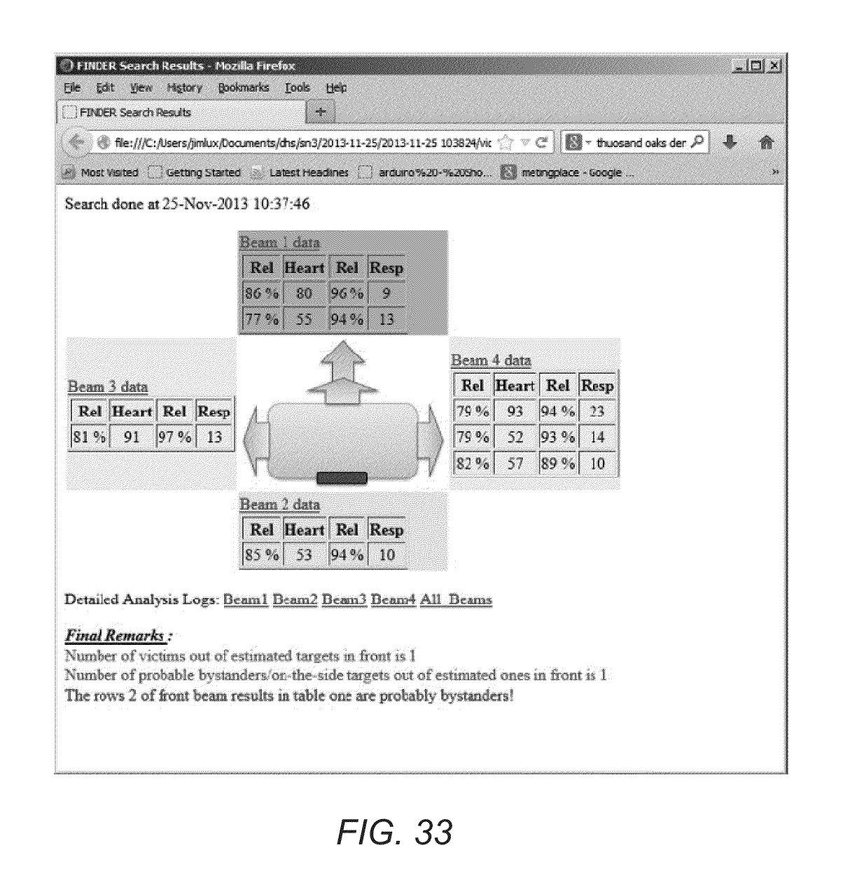

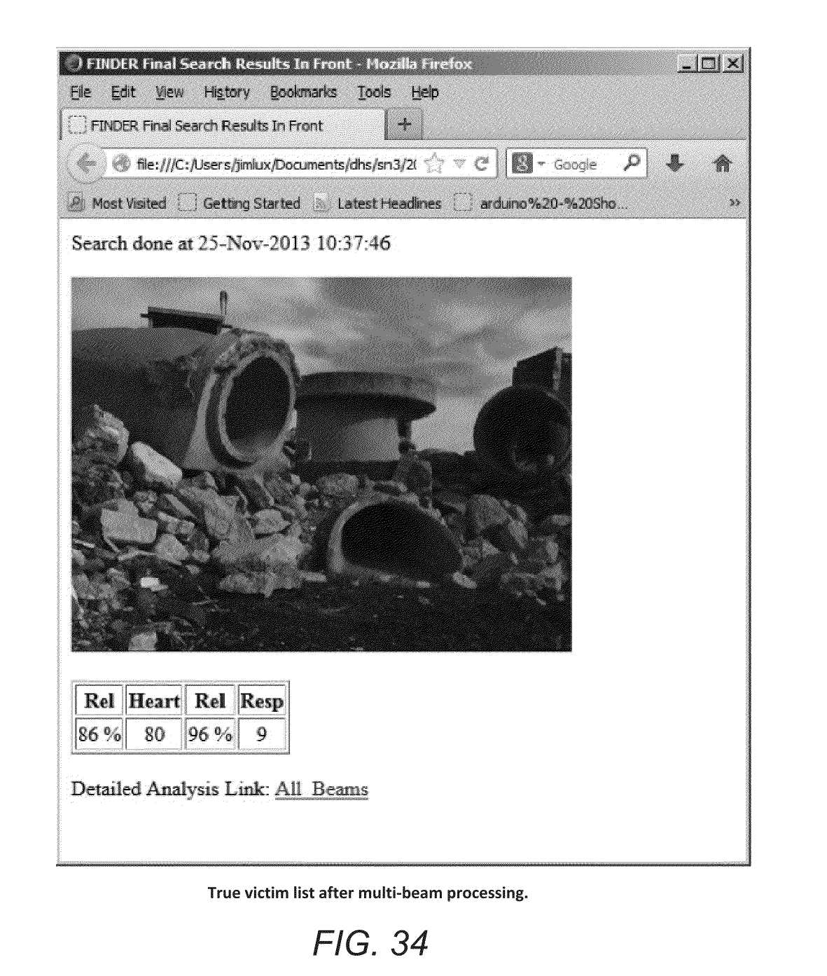

An example of sample detected targets and output from multi-beam processing is illustrated in FIG. 33. An example of a true victim list after multi-beam processing in accordance with embodiments of the invention is illustrated in FIG. 34. Although FIGS. 33-34 illustrate an example of the user interface for displaying information regarding detected outputs, any of a variety of different user interfaces may be implemented as appropriate to the requirements of specific applications for displaying information in accordance with embodiments of invention.

### Software Module Structure

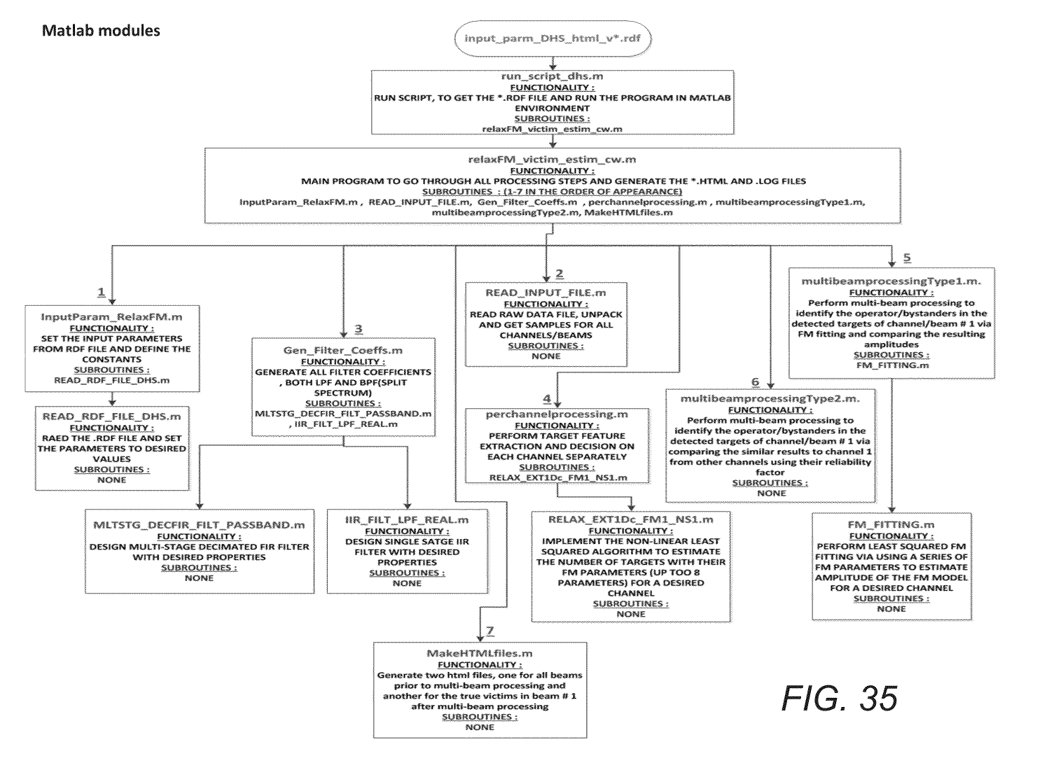

An example of the structure of the software modules and their corresponding subroutines as well as functionality used for signal processing in accordance with embodiments of the invention is illustrated in FIG. 35. Note that the numbers in this figure imply the order of appearance for each module in the main module. Although FIG. 35 illustrates an example of the structure of the software modules, any variety of software modules may be implemented as appropriate to the requirements of specific applications in accordance with embodiments of the invention.

While the above description contains many specific embodiments of the invention, these should not be construed as limitations on the scope of the invention, but rather as an example of one embodiment thereof It is therefore to be understood that the present invention may be practiced otherwise than specifically described, without departing from the scope and spirit of the present invention. Thus, embodiments of the present invention should be considered in all respects as illustrative and not restrictive.

What is claimed is:

1. An integrated microwave sensor module comprising:
a transmitter unit comprising a variable frequency microwave source connected to at least one transmitter unit amplifier, where:
the variable frequency microwave source is configured to generate at least one continuous wave (“CW”) transmit signal based upon at least one frequency control signal received from a microcontroller unit; and
the at least one transmitter unit amplifier is configured to receive and amplify the at least one CW transit signal;
a receiver unit configured to receive at least one return signal and utilize a cancellation path to cancel contributions to the return signal that are not the result of reflections from a target;
a microcontroller unit configured to communicate with the transmitter and receiver units comprising:
a processor;
a memory containing a microcontroller application, wherein the microcontroller application configures the processor to:
split the return signal into a heartbeat band and a breathing band using bandpass filtering;
perform parameter estimation for the breathing band using a non-least squares process (NLS);
perform parameter estimation for the heartbeat band using an NLS process;
analyze breathing and heartbeat results based on the heartbeat FM frequency and the breathing center frequency; and
output detected targets based on analysis.

2. The integrated microwave sensor module of claim 1, wherein a NLS process fits a complex input to a frequency modulated (FM) model.

3. The integrated microwave sensor module of claim 1, wherein the microcontroller application further configures the processor to low pass filter and decimate the return signal.

4. The integrated microwave sensor module of claim 1, wherein the microcontroller application further configures the processor to remove a linear trend from the return signal using a linear least square fitting in the data.

5. The integrated microwave sensor module of claim 1, wherein the microcontroller application further configures the processor to remove signals that are out of band.

6. The integrated microwave sensor module of claim 1, wherein the microcontroller application further configures the processor to identify and remove 2^{nd} and 3^{rd} harmonics from a list of detected frequencies of the breathing band.

7. The integrated microwave sensor module of claim 1, wherein the microcontroller application further configures the processor to remove harmonics of breathing signals that appear in the heart band.

8. The integrated microwave sensor module of claim 1, wherein the microcontroller application further configures the processor to remove out of band heartbeat signals from the heart band by removing targets whose center frequency is out of an assigned bandwidth.

9. The integrated microwave sensor module of claim 1, wherein the microcontroller application further configures the processor to remove targets whose FM frequency is out of a particular FM frequency range.

10. The integrated microwave sensor module of claim 1, wherein the microcontroller application further configures the processor to remove targets whose relative amplitude with respect to a maximum in the heartbeat band is below a certain threshold based on a dynamic range for detected targets.

11. The integrated microwave sensor module of claim 1, wherein the microcontroller application further configures the processor to remove targets whose relative amplitude with respect to a maximum in the breathing band is below a certain threshold based on a desired dynamic range for detected targets.

12. The integrated microwave sensor module of claim 1, wherein the microcontroller application further configures the processor to remove heart signals whose relative amplitude with respect to breathing is large within a certain threshold.

13. The integrated microwave sensor module of claim 1, wherein the microcontroller application further configures the processor to match breathing results with heartbeat results based on the heartbeat FM frequency and breathing center frequency.

14. The integrated microwave sensor module of claim 1, wherein the microcontroller application further configures the processor to calculate reliability factors for the heartbeat band and the breathing band by using corresponding signal to noise ratio “SNR” values.

15. The integrated microwave sensor module of claim 1, wherein the microcontroller application further configures the processor to compare a plurality of signals received from a plurality of receivers to identify false targets.

16. The integrated microwave sensor module of claim 1, wherein the microcontroller application further configures the processor to compare results from each of a plurality of received signals to identify false targets.

17. A method of detecting a target using a life detecting radar, the method comprising:
propagating at least one beam using a continuous wave transmit signal set at a plurality of frequencies, where the at least one beam illuminates at least one sensing area using at least one transmit unit;
receiving a return signal associate with reflections of the at least one transmit signal from objects within the at least one sensing area using at least one receive antenna;
receiving the return signal from the at least one receive antenna using a life detecting radar system;
splitting the return signal into a heartbeat band and a breathing band using bandpass filtering;
performing parameter estimation for the breathing band using a non-least squares process (NLS);
performing parameter estimation for the heartbeat band using an NLS process;
analyzing breathing and heartbeat results based on the heartbeat FM frequency and the breathing center frequency; and
outputting detected targets based on analysis.

18. The method of claim 17, wherein a NLS process fits a complex input to a frequency modulated (FM) model.

19. The method of claim 17, further comprising applying a low pass filter to the return signal.

20. The method of claim 17, further comprising removing a linear trend from the return signal using a linear least square fitting in the data.
